(19) United States
(12) Patent Application Publication
(10) Pub. No.: US 2025/0353811 A1
(43) Pub. Date: Nov. 20, 2025

(54) A CONTINUOUS FLOW PROCESS FOR SYNTHESIS OF ORGANIC AZIDES

(71) Applicant: INDIAN INSTITUTE OF SCIENCE EDUCATION AND RESEARCH, PUNE (IISER PUNE), Pune, Maharashtra (IN)

(72) Inventors: Boopathy Gnanaprakasam, Pune, Maharashtra (IN); Akanksha M. Pandey, Pune, Maharashtra (IN); Shankhajit Mondal, Pune, Maharashtra (IN)

(21) Appl. No.: 18/871,215

(22) PCT Filed: Jun. 2, 2023
(86) PCT No.: PCT/IB2023/055692

§ 371 Date: Dec. 3, 2024

(30) Foreign Application Priority Data
Jun. 3, 2022 (IN) 202221031963

(51) Int. Cl. C07C 247/06; A61K 31/357; A61K 31/404; A61K 31/4192; A61K 31/435; A61K 31/498; A61K 31/538; A61K 31/655; C07C 247/14; C07D 209/40; C07D 219/02; C07D 241/44; C07D 249/04; C07D 265/36; C07D 317/52

(52) U.S. Cl. CPC C07C 247/06; A61K 31/357; A61K 31/404; A61K 31/4192; A61K 31/435; A61K 31/498; A61K 31/538; A61K 31/655; C07C 247/14; C07D 209/40; C07D 219/02; C07D 241/44; C07D 249/04; C07D 265/36; C07D 317/52

(57) ABSTRACT

The present disclosure relates generally to the field of synthetic organic chemistry. More specifically, the disclosure is directed to a continuous flow process for synthesis of azides from alcohols and peroxides, wherein the process comprises azidation with trimethylsilyl azide and a catalyst Amberlyst-15. It is a non-hazardous, mild and controlled reaction giving good yields of azides. The azides can be further employed for synthesis of medicinally and industrially useful chemicals.

### FIELD OF THE INVENTION

The present disclosure relates generally to the field of synthetic organic chemistry. More specifically, the disclosure is directed to a continuous flow process for synthesis of azides from alcohols and peroxides, wherein the process comprises azidation with trimethylsilyl azide and a catalyst Amberlyst-15.

### BACKGROUND OF THE INVENTION

Background description includes information that may be useful in understanding the present invention. It is not an admission that any of the information provided herein is prior art or relevant to the presently claimed invention, or that any publication specifically or implicitly referenced is prior art.

Nitrogen containing heterocyclic compounds has shown widespread utility in pharmaceutical applications. Azides are compounds with the formula N₃ of use in for example in rocket propellants. Besides the usefulness of azides, many 1, 2, 3- or 1, 2-nitrogen enriched heterocycles were synthesized from organic azides or hydrazides through click reactions or condensation chemistry. For instance, triazole has been a very important heterocycle in many antifungal applications and other diseases. Other heterocycles such as 2H-1, 4-benzoxazin-3(4H)-one and quinoxaline-2(1H)-ones also have proven applications in medicinal chemistry. Many organic intermediates have shown intriguing reactions to generate the respective products with greater molecular complexity.

In 19^{th} century, azides, being an indispensable tool for performing various chemical operations, witnessed an impressive library of powerful named reactions (The Chemistry of the Azido Group (Ed.: S. Patai), Wiley, New York, 1971; The Chemistry of Halides, Pseudo-halides and Azides, Supplement D, (Eds.: S. Patai, Z. Rappoport), Wiley, Chichester, 1983; Chemistry of Halides, Pseudo-Halides and Azides, Part 1 (Ed.: S. Patai), Wiley, Chichester, 1995; Chemistry of Halides, Pseudo-Halides and Azides, Part 2 (Ed.: S. Patai), Wiley, Chichester, 1995; Monograph: Azides and Nitrenes Reactivity and Utility (Ed.: E. F. V. Scriven), Academic Press, New York, 1984). These energy rich intermediates are building blocks for the bio-conjugation of proteins (Jang, S.; Sachin, K.; Lee, H.; Wook Kim, D.; Soo Lee, H. Development of a Simple Method for Protein Conjugation by Copper-Free Click Reaction and Its Application to Antibody-Free Western Blot Analysis. Bioconjugate Chem. 2012, 23, 2256-2261.). These are readily converted into N-heterocycles, (Brase, S.; Gil, C.; Knepper, K.; Zimmermann, V. Organic Azides: An Exploding Diversity of a Unique Class of Compounds. Angew. Chem., Int. Ed. 2005, 44, 5188-5240; Padwa, A. Aziridines and Azirines: Monocyclic. In Comprehensive Heterocyclic Chemistry III; Katritzky, A. R., Ramsden, C. A., Scriven, E. F. V., Taylor, R. J. K., Eds.; Elsevier Science: Oxford, 2008; Vol. 1, Chapter 1.01.6.2, pp 50-64) and also known as effective ammonia surrogates (Gololobov, Y. G.; Kasukhin, L. F. Recent Advances in the Staudinger Reaction. Tetrahedron 1992, 48, 1353-1406). Moreover, organic azides were also used for (3+2) cycloaddition with alkynes and nitrile to generate triazole and tetrazole moiety to access important bioactive molecules such as anticancer, antimicrobial drug and as an aldose reductase inhibitor (Li, Y. L.; Combs, A. P. Bicyclic Heteroaryl amino alkyl Phenyl Derivatives as PI3K Inhibitors. Int. Patent Appl. WO2015191677A1, Dec. 17, 2015; Kim, M. S.; Yoo, M. H.; Rhee, J. K.; Kim, Y. J.; Park, S. J.; Choi, J. H.; Sung, S. Y.; Lim, H. G.; Cha, D. W. Synthetic Intermediates, Process for Preparing Pyrrolylheptanoic Acid Derivatives Therefrom. Int. Patent Appl. WO2009084827A2, Jul. 9, 2009; Bathula, S. N. V. P.; Vadla, R. Bioactivity of 1, 4-disubstituted 1, 2, 3-triazoles as Cytotoxic Agents Against the Various Human Cell Lines. Asian J. Pharm. Clin. Res. 2011, 4, 66-67; Aganda, K. C.; Hong, B.; Lee, A. Visible-Light-Promoted Switchable Synthesis of C-3-Functionalized Quinoxalin-2(1H)-ones. Adv. Synth. Catal. 2021, 363, 1443-1448). Albeit its exponential application in chemical transformation towards biological applications, it possesses severe concerns on the safety due to its explosive nature in large-scale manufacturing process. Moreover, azides with C/N≥3 are generally stable to handle. (Brase, S.; Banert, K., Eds. Organic Azides: Syntheses and Applications; John Wiley & Sons, Ltd.: Chichester, U.K., 2010.). Hence, a process technology is required to augment the safety concern in azide synthesis and relevant associated chemical transformations.

For the synthesis of alkyl azides the traditional batch methods involve the activation of —OH group which requires two steps: i) conversion to a good leaving group; and ii) substitution reaction with NaN₃. After activation of —OH group, it can be converted into genotoxic alkyl halide (Li, J.; Cao, J.; Wei, J.; Shi, X.; Zhang, L.; Feng, J.; Chen, Z. Ionic Liquid Brush as a Highly Efficient and Reusable Catalyst for On-Water Nucleophilic Substitutions. Eur. J. Org. Chem. 2011, 2011, 229-233), sulfonates (Denk, C.; Wilkovitsch, M.; Skrinjar, P.; Svatunek, D.; Mairinger, S.; Kuntner, C.; Filip, T.; Fröhlich, J.; Wanek, T.; Mikula, H. [18F] Fluoroalkyl Azides for Rapid Radiolabeling and (Re) investigation of their Potential Towards in vivo Click Chemistry. Org. Biomol. Chem. 2017, 15, 5976-5982) or acetates (Kurosawa, W.; Kan, T.; Fukuyama, T. Stereocontrolled Total Synthesis of (−)-Ephedradine A (Orantine). J. Am. Chem. Soc. 2003, 125, 8112-8113) which on reaction with NaN₃ afford azides. In addition, it can also be accessed using other precursors such as amines, hydrazines, etc. However, the tedious workup and safety concern in scale up of the reaction becomes a substantial challenge. Hence developing a direct azidation approach is the best way to avoid waste generation and also minimize the synthetic steps. To this credit, Mitsunobu reaction shows direct substitution of the hydroxyl group to attain azides using hydrazoic acid (Besset, C.; Chambert, S.; Fenet, B.; Queneau, Y. Direct Azidation of Unprotected Carbohydrates under Mitsunobu Conditions using Hydrazoic Acid. Tetrahedron Lett. 2009, 50, 7043-7047). However, in view of potential safety concerns related with genotoxic sodium azide and hydrazoic acid a safe and practical azide source needs to be investigated with new methodologies. Apart from this, azides are also generated using various Lewis acid catalysts such as BF₃·OEt₂, NaAuCl₄, Cu(OTf)₂, AgOTf, FeCl₃, MoCl₅, InBr₃, and Bi(OTf)₃ which facilitate the substitution by the activation of the hydroxyl group (Terrasson, V.; Marque, S.; Georgy, M.; Campagne, J. M.; Prim, D. Lewis Acid-Catalyzed Direct Amination of Benzhydryl Alcohols. Adv. Synth. Catal. 2006, 348, 2063-2067; Khedar, P.; Pericherla, K.; Kumar, A. Copper Triflate: An Efficient Catalyst for Direct Conversion of Secondary Alcohols into Azides. Synlett 2014, 25, 515-518; Rueping, M.; Vila, C.; Uria, U. Direct Catalytic Azidation of Allylic Alcohols. Org. Lett. 2012, 14, 768-771; Sawama, Y.; Nagata, S.; Yabe, Y.; Morita, K.; Monguchi, Y.; Sajiki, H. Iron-Catalyzed Chemoselective Azidation of Benzylic Silyl Ethers. Chem. Eur. J. 2012, 18, 16608-16611; Reddy, C. R.; Madhavi, P. P.; Reddy, A. S. Molybdenum (V) Chloride-Catalyzed Amidation of Secondary Benzyl Alcohols with Sulfonamides and Carbamates. Tetrahedron Lett. 2007, 48, 7169-7172; Kumar, A.; Sharma, R. K.; Singh, T. V.; Venugopalan, P. Indium (III) Bromide Catalyzed Direct Azidation of α-hydroxyketones using TMSN3. Tetrahedron 2013, 69, 10724-10732; Tummatorn, J.; Thongsornkleeb, C.; Ruchirawata, S.; Thongarama, P.; Kaewmee, B. Convenient and Direct Azidation of Sec-Benzyl Alcohols by Trimethylsilyl Azide with Bismuth (III) Triflate Catalyst. Synthesis 2015, 47, 323-329). However, contrary to Lewis acid-mediated azidation reactions, less approaches have been accomplished for this transformation using Brønsted acid catalyst. Hajipour used acidic ionic liquid [H-NMP]HSO₄, for this transformation using alcohols and sodium azide (Hajipour, A. R.; Rajaei, A.; Ruoho, A. E. A Mild and Efficient Method for Preparation of Azides from Alcohols using Acidic Ionic Liquid [H-NMP] HSO₄. Tetrahedron Lett. 2009, 50, 708-711) whereas Onaka demonstrated a combination of TMSCl and TMSN₃ with montmorillonite clay to get azides (Tandiary, M. A.; Masui, Y.; Onaka, M. A Combination of Trimethylsilyl Chloride and Hydrous Natural Montmorillonite Clay: An Efficient Solid Acid Catalyst for the Azidation of Benzylic and Allylic Alcohols with Trimethylsilyl Azide. RSC Adv. 2015, 5, 15736-15739). Similarly, Rode accomplished it with the use of a solid povidone and phosphotungstic acid hybrid as a catalyst for heterogeneous azidation of alcohols (Kamble, S.; More, S.; Rode, C. Highly Selective Direct Azidation of Alcohols Over a Heterogeneous Povidone-Phosphotungstic Solid Acid Catalyst. New J. Chem. 2016, 40, 10240-10245). More recently, Zhou and Regier demonstrated it with aqueous perchloric acid (Yin, X. P.; Zhu, L.; Zhou, J. Metal-Free Azidation of α-Hydroxy Esters and α-Hydroxy Ketones Using Azidotrimethylsilane. Adv. Synth. Catal. 2018, 360, 1116-1122) and HBF₄·OEt₂ (Regier, J.; Maillet, R.; Bolshan, Y. A Direct Brønsted Acid Catalyzed Azidation of Benzhydrols and Carbohydrates. Eur. J. Org. Chem. 2019, 2390-2396) respectively. Although numerous methods exist for azidation, still more convenient methods for the safer generation of azide are highly desired.

In order to minimize the safety hazards associated with reaction scale-up of these explosive and high energy molecules which decompose with heat, light, shock under batch conditions continuous flow methods may be explored. The potential of continuous flow for azidation has been explored by using imidazole-1-sulfonyl azide hydrochloride as diazotransfer reagent for benzyl amine to azide transfer reaction (Delvillea, M.; Nieuwland, P.; Janssena, P.; Koch, K.; Van Hest, J.; Rutjes, F. Continuous Flow Azide Formation: Optimization and Scale-up. Chem. Eng. J. 2011, 167, 556-559) and aqueous sodium azide for C-3 azidation of mesyl shikimate (Sagandira, C.; Watts, P. Safe and Highly Efficient Adaptation of Potentially Explosive Azide Chemistry Involved in the Synthesis of Tamiflu Using Continuous-Flow Technology. Beilstein J. Org. Chem. 2019, 15, 2577-2589). Furthermore, azidation with azide exchange resin was a crucial step in total synthesis of oxomaritidine (Baxendale, I.; Deeley, J.; Griffiths-Jones, C.; Ley, S.; Saaby, S.; Tranmer, G. A Flow Process for the Multi-Step Synthesis of the Alkaloid Natural Product Oxomaritidine: A New Paradigm for Molecular Assembly. Chem. Commun. 2006, 2566-2568). Moreover, telescoped flow process was also established to get propargyl amine using DPPA (Donnelly, A.; Zhang, H.; Baumann, M. Development of a Telescoped Flow Process for the Safe and Effective Generation of Propargylic. Molecules 2019, 24, 3658). However, these methods either used NaN₃ or severe heating conditions.

Thus, there is a need in the art to develop a mild, safe and efficient process of synthesis of azides from a wide range of substrates which are suitable for large scale synthesis and do not use toxic reagents.

### Objects of the Invention

An objective of the present disclosure is to provide a continuous flow process for a synthesis of azides that is a direct azidation process.

Another objective of the present disclosure is to provide a continuous flow process for the synthesis of azides which is easily scaled up and does not employ toxic reagents.

Another objective of the present disclosure is to provide a process for the synthesis of azides from alcohols and peroxides with high yield.

### SUMMARY OF THE INVENTION

This summary is provided to introduce a selection of concepts in a simplified form that are further described below in the detailed Description section. This summary is not intended to identify key features or essential features of the claimed subject matter, nor is it intended to be used as an aid in determining the scope of the claimed subject matter.

Aspects of the present disclosure provide a process of synthesizing azides from different alcohols and peroxides by a continuous flow method that is safe and mild.

In an aspect, the present disclosure provides a process of synthesizing organic azides of formula (I) by direct azidation of alcohols of Formula (II).

In an embodiment, the present disclosure provides a continuous flow process of synthesizing organic azides of formula (I), a stereoisomer, a tautomer, a pharmaceutically acceptable salt or a pharmaceutically acceptable solvate thereof, wherein the process comprises the step of reacting a compound of formula (II) with trimethylsilyl azide and a catalyst Amberlyst-15;

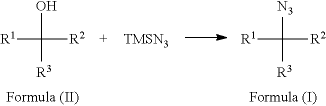

wherein R¹ is selected from substituted or unsubstituted (C₆₋₁₆)aryl, or substituted or unsubstituted (C₅₋₁₀) heterocycle;
wherein R² and R³ are independently selected from H, substituted or unsubstituted (C₆₋₁₆)aryl, substituted or unsubstituted (C₁₋₆)alkyl, or substituted or unsubstituted —CH₂—(C₆₋₁₆)aryl; and
wherein the substituents may be selected from one or more of halogen, (C₁₋₆)alkyl, cyano, nitro, —NH₂, (C₁₋₆) alkoxy, —COOH, or combinations thereof.

In a preferred embodiment, the compound of formula (I) may be selected from:
(azidomethylene)dibenzene;
1-(azido(phenyl)methyl)-4-chlorobenzene;
4,4′-(azidomethylene)bis(methoxybenzene);
(1-azidoethyl)benzene;
2-(1-azidoethyl)naphthalene;
(1-azidoethane-1,1-diyl)dibenzene;
(azido methanetriyl)tribenzene;
5-(azidomethyl)benzo[d][1,3]dioxole;
5-(azidomethyl)-6-chlorobenzo[d][1,3]dioxole;
4-(azidomethyl)pyrene;
   a stereoisomer, a tautomer, a pharmaceutically acceptable salt or a pharmaceutically acceptable solvate thereof.

In an aspect, the present disclosure provides a process of synthesizing organic azides by direct azidation of 3-hydroxy-2-oxindole compounds.

In an embodiment, the present disclosure provides a continuous flow process of synthesizing organic azides of formula (III), a stereoisomer, a tautomer, a pharmaceutically acceptable salt or a pharmaceutically acceptable solvate thereof, wherein the process comprises the step of reacting a compound of formula (IV) with trimethylsilyl azide and a catalyst Amberlyst-15;

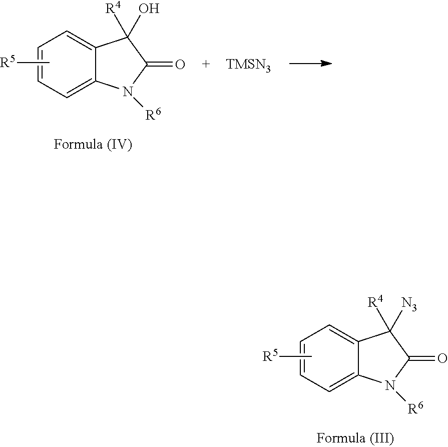

wherein R⁴, R⁵ and R⁶ are independently selected from one or more of H, halogen, —COOH, nitro, —NH₂, (C₁₋₆)alkoxy, substituted or unsubstituted (C₆₋₁₆)aryl, substituted or unsubstituted (C₁₋₆)alkyl; or substituted or unsubstituted —CH₂—(C₆₋₁₆)aryl; and
wherein the substituents may be selected from one or more of halogen, (C₁₋₆)alkyl, cyano, nitro, —NH₂, —COOH, (C₁₋₆)alkoxy, or combinations thereof.

In a preferred embodiment, the compounds of formula (III) may be selected from:
3-azido-3-methyl indolin-2-one;
3-azido-3-phenyl indolin-2-one;
3-azido-3-(p-tolyl)indolin-2-one;
3-azido-3-(4-methoxyphenyl)indolin-2-one;
3-azido-3-benzyl indolin-2-one;
3-azido-3-(3,4-dimethoxybenzyl)indolin-2-one;
3-azido-3-(4-bromobenzyl)indolin-2-one;
3-azido-3-benzyl-6-chloro indolin-2-one;
3-azido-1,3-dibenzyl indolin-2-one;
3-azido-1,3-dimethyl indolin-2-one;
   a stereoisomer, a tautomer, a pharmaceutically acceptable salt or a pharmaceutically acceptable solvate thereof.

In an aspect, the present disclosure provides a process of synthesizing organic 2-azido-2H-benzo[b][1,4]oxazin-3(4H)-one derivatives from peroxyoxyindoles.

In an embodiment, the present disclosure provides a continuous flow process of synthesizing organic azides of formula (V), a stereoisomer, a tautomer, a pharmaceutically acceptable salt or a pharmaceutically acceptable solvate thereof, wherein the process comprises the step of reacting a compound of formula (VI) with trimethylsilyl azide and a catalyst Amberlyst-15.

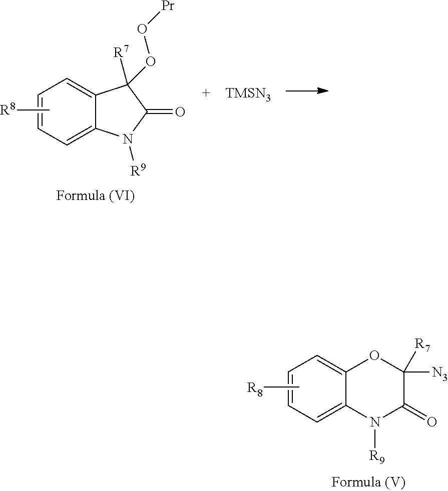

wherein R⁷ may be selected from H, (C₁₋₆)alkyl, substituted or unsubstituted (C₆₋₁₆)aryl; or substituted or unsubstituted —CH₂—(C₆₋₁₆)aryl;
wherein R⁸ and R⁹ may be independently selected from one or more of H, halogen, (C₁₋₆)alkyl, cyano, nitro, (C₁₋₆)alkoxy, substituted or unsubstituted —CH₂—(C₆₋₁₆)aryl, substituted or unsubstituted (C₆₋₁₆)aryl or combinations thereof;
wherein the substituent may be selected from one or more of halogen, (C₁₋₆)alkyl, cyano, nitro, (C₁₋₆)alkoxy, —COOH, —NH₂ or combinations thereof; and
wherein Pr is a protecting group.

In a preferred embodiment, the compounds of formula (V) may be selected from:
2-azido-2-benzyl-2H-benzo[b][1,4]oxazin-3(4H)-one;
2-azido-2-methyl-2H-benzo[b][1,4]oxazin-3(4H)-one;
2-azido-2-(4-methoxyphenyl)-2H-benzo[b][1,4]oxazin-3(4H)-one;
2-azido-2-(2-fluorobenzyl)-2H-benzo[b][1,4]oxazin-3(4H)-one;
2-azido-2-(4-bromobenzyl)-2H-benzo[b][1,4]oxazin-3(4H)-one;
2-azido-2-benzyl-6-chloro-2H-benzo[b][1,4]oxazin-3(4H)-one;
2-azido-2-(4-bromobenzyl)-6-chloro-2H-benzo[b][1,4]oxazin-3(4H)-one;
2-azido-6-chloro-2-(4-methylbenzyl)-2H-benzo[b][1,4]oxazin-3(4H)-one;
2-azido-2,4-dimethyl-2H-benzo[b][1,4]oxazin-3(4H)-one;
2-azido-4-benzyl-2-methyl-2H-benzo[b][1,4]oxazin-3(4H)-one;
   a stereoisomer, a tautomer, a pharmaceutically acceptable salt or a pharmaceutically acceptable solvate thereof.

In an aspect, the present disclosure provides a process of synthesizing organic azides by direct azidation of 9-alkyl/aryl-9H-fluoren-9-ol derivatives.

In an embodiment, the present disclosure provides a continuous flow process of synthesizing organic azides of formula (I′), a stereoisomer, a tautomer, a pharmaceutically acceptable salt or a pharmaceutically acceptable solvate thereof, wherein the process comprises the step of reacting a compound of formula (II′) with trimethylsilyl azide and a catalyst Amberlyst-15;

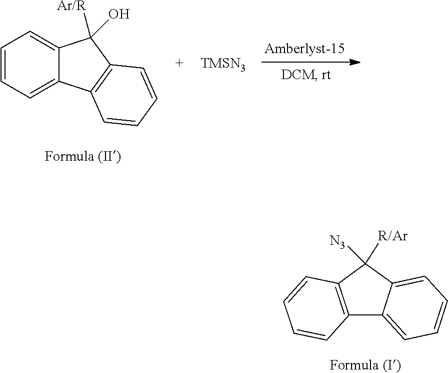

wherein Ar/R is selected from group consisting of substituted or unsubstituted (C₆₋₁₆)aryl, or substituted or unsubstituted (C₅₋₁₀)heterocycle substituted or unsubstituted (C₆₋₁₆)aryl, substituted or unsubstituted (C₁₋₆)alkyl, or substituted or unsubstituted —CH₂—(C₆₋₁₆)aryl; and one or more of halogen, (C₁₋₆)alkyl, cyano, nitro, —NH₂, (C₁₋₆)alkoxy, —COOH, or combinations thereof.

In a preferred embodiment, the compound of formula (I′) may be selected from:
9-azido-9-phenyl-9H-fluorene;
9-azido-9-(p-tolyl)-9H-fluorene;
9-azido-9-(4-methoxyphenyl)-9H-fluorene;
9-([1,1′-biphenyl]-4-yl)-9-azido-9H-fluorene;
9-azido-9-hexyl-9H-fluorene;
9-azido-2,7-dibromo-9-(4-methoxyphenyl)-9H-fluorene;
9-azido-9-benzyl-9H-fluorene;
9-azido-9-(3-phenoxybenzyl)-9H-fluorene;
9-([1,1′-biphenyl]-4-ylmethyl)-9-azido-9H-fluorene;
9-azido-9-(4-methoxybenzyl)-9H-fluorene;
9,9′-diazido-9H,9′H-9,9′-bifluorene;
9-azido-2-bromo-9-phenyl-9H-fluorene,
9-azido-2,7-dibromo-9-phenyl-9H-fluorene;
9-azido-2,7-dibromo-9-(p-tolyl)-9H-fluorene; or
   a stereoisomer, a tautomer, a pharmaceutically acceptable salt or a pharmaceutically acceptable solvate thereof.

Other aspects of the invention will be set forth in the description which follows, and in part will be apparent from the description, or may be learnt by the practice of the invention.

### DETAILED DESCRIPTION OF THE INVENTION

The following is a detailed description of embodiments of the disclosure. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In some embodiments, numbers have been used for quantifying amounts, percentages, ratios, and so forth, to describe and claim certain embodiments of the invention and are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be constructed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Various terms as used herein are shown below. To the extent a term used in a claim is not defined below, it should be given the broadest definition persons in the pertinent art have given that term as reflected in printed publications and issued patents at the time of filing.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Unless the context requires otherwise, throughout the specification which follow, the word “comprise” and variations thereof, such as, “comprises” and “comprising” are to be construed in an open, inclusive sense that is as “including, but not limited to.”

The recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified.

The description that follows, and the embodiments described therein, is provided by way of illustration of an example, or examples, of particular embodiments of the principles and aspects of the present disclosure. These examples are provided for the purposes of explanation, and not of limitation, of those principles and of the disclosure.

It should also be appreciated that the present disclosure can be implemented in numerous ways, including as a system, a method or a device. In this specification, these implementations, or any other form that the invention may take, may be referred to as processes. In general, the order of the steps of the disclosed processes may be altered within the scope of the invention.

The headings and abstract of the invention provided herein are for convenience only and do not interpret the scope or meaning of the embodiments.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

The term “or”, as used herein, is generally employed in its sense including “and/or” unless the content clearly dictates otherwise.

The term, “(C₁₋₆) alkyl”, as used herein, refers to saturated aliphatic groups, including straight or branched-chain alkyl groups having six or fewer carbon atoms in its backbone, for instance, C₁₋₆ for straight chain and C₃-C₆ for branched chain. As used herein, (C₁₋₆) alkyl refers to an alkyl group having from 1 to 6 carbon atoms. Representative examples of alkyl include, but are not limited to, methyl, ethyl, n-propyl, n-butyl, n-pentyl, n-hexyl, isopropyl, sec-butyl, isobutyl, tert-butyl, isopentyl, 2-methylbutyl and 3-methylbutyl.

Furthermore, unless stated otherwise, the alkyl group can be unsubstituted or substituted with one or more substituents, for example, from one to four substituents, independently selected from the group consisting of halogen, hydroxy, cyano, nitro and amino. Examples of substituted alkyl include, but are not limited to hydroxymethyl, 2-chlorobutyl, trifluoromethyl and aminoethyl.

The term “(C₁₋₆)alkoxy” refers to a (C₁₋₆)alkyl having an oxygen attached thereto. Representative examples of alkoxy groups include, but are not limited to, methoxy, ethoxy, propoxy, isopropoxy, n-butoxy, isobutoxy and tert-butoxy. Furthermore, unless stated otherwise, the alkoxy groups can be unsubstituted or substituted with one or more groups. A substituted alkoxy refers to a (C₁₋₆)alkoxy substituted with one or more groups, particularly one to four groups independently selected from the groups indicated above as the substituents for the alkyl group.

The term “(C₆₋₁₆) aryl” or “aryl” as used herein refers to monocyclic, bicyclic, tricyclic or tetracyclic hydrocarbon groups having 6 to 16 ring carbon atoms, wherein at least one carbocyclic ring is having a π electron system. Examples of (C₆-C₁₆) aryl ring systems include, but are not limited to, phenyl, pyrenyl, or naphthyl. Unless indicated otherwise, aryl group can be unsubstituted or substituted with one or more substituents, for example 1-4 substituents independently selected from the group consisting of halogen, (C₁₋₆)alkyl, hydroxy, cyano, nitro, —COOH, amino and (C₁₋₆)alkoxy.

The term, (C₅₋₁₀)heterocycle, as used herein refers to a 5- to 10-membered, saturated, partially unsaturated or unsaturated monocyclic or bicyclic ring system containing 1 to 4 heteroatoms independently selected from the group consisting of oxygen, nitrogen and sulfur. Saturated heterocyclic ring systems do not contain any double bond, whereas partially unsaturated heterocyclic ring systems contain at least one double bond, and unsaturated heterocyclic ring systems form an aromatic system containing heteroatom(s). The oxidized form of the ring nitrogen and sulfur atom contained in the heterocycle to provide the corresponding N-oxide, S-oxide or S, S-dioxide is also encompassed in the scope of the present invention. Representative examples of heterocycles include, but are not limited to, tetrahydrofuran, tetrahydrothiophene, pyrrolidine, dihydropyran, tetrahydropyran, thio-dihydropyran, thio-tetrahydropyran, piperidine, piperazine, morpholine, 1,3-oxazinane, 1,3-thiazinane, 4,5,6-tetrahydropyrimidine, 2,3-dihydrofuran, dihydrothiene, dihydropyridine, tetrahydropyridine, isoxazolidine, pyrazolidine, furan, pyrrole, thiophene, imidazole, oxazole, thiazole, triazole, tetrazole, benzofuran, indole, benzoxazole, benzodioxole, benzothiazole, isoxazole, triazine, purine, pyridine, pyrazine, quinoline, isoquinoline, phenazine, oxadiazole, pteridine, pyridazine, quinazoline, pyrimidine, isothiazole, benzopyrazine andtetrazole. Unless stated otherwise, (C₅₋₁₀)heterocycles can be unsubstituted or substituted with one or more substituents, for example, substituents independently selected from the group consisting of oxo, halogen, hydroxy, cyano, nitro, amine, (C₁₋₆)alkyl and COOH.

The term, “halogen” as used herein refers to chlorine, fluorine, bromine or iodine atoms.

Aspects of the present disclosure provide a continuous flow synthesis of organic azides that streamlines the assembly and delivery of reactants and products by mitigating safety concerns.

In an embodiment, the present disclosure provides a process of synthesis of organic azides from substrates such as alcohols including primary, secondary and tertiary alcohols and peroxides using trimethylsilyl azide (TMSN₃) as an azide transfer agent; wherein the process is a continuous flow process.

In an embodiment, the present disclosure provides a process of synthesis of organic azides by direct azidation that employs the environmentally non-hazardous and industrially benefitted Amberlyst-15 as catalyst.

In an embodiment, the present disclosure provides a process of synthesizing organic azides of Formula (I) by direct azidation of alcohols of Formula (II).

In an embodiment, the present disclosure provides a continuous flow process of synthesizing organic azides of formula (I), a stereoisomer, a tautomer, a pharmaceutically acceptable salt or a pharmaceutically acceptable solvate thereof, wherein the process comprises the step of reacting a compound of formula (II) with trimethylsilyl azide and a catalyst Amberlyst-15 in a suitable solvent;

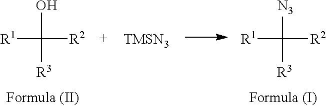

wherein R¹ is selected from substituted or unsubstituted (C₆₋₁₆)aryl, or substituted or unsubstituted (C₅₋₁₀)heterocycle;
wherein R² and R³ are independently selected from H, substituted or unsubstituted (C₆₋₁₆)aryl, substituted or unsubstituted (C₁₋₆)alkyl, or substituted or unsubstituted —CH₂—(C₆₋₁₆)aryl; and wherein the substituents may be selected from one or more of halogen, (C₁₋₆) alkyl, cyano, nitro, —NH₂, (C₁₋₆)alkoxy, —COOH, or combinations thereof.

In an embodiment, R¹ is substituted or unsubstituted (C₆₋₁₆)aryl selected from phenyl, naphthyl, or pyrenyl; or a substituted or unsubstituted (C₅₋₁₀) heterocycle selected from 1,3-benzodioxole; wherein the substituents may be one or more of —OCH₃, or halogen.

In an embodiment, R² and R³ are independently selected from H, substituted or unsubstituted phenyl, or —CH₃; wherein the substituents may be selected from one or more of halogen, or —OCH₃.

In an embodiment, the ratio of formula (II) to TMSN₃ may be in the range of about 1:10 to about 10:1, preferably about 1:3.

In an embodiment, the present disclosure provides a compound of Formula (I), a stereoisomer, a tautomer, a pharmaceutically acceptable salt or a pharmaceutically acceptable solvate thereof obtained by the process as recited above.

In a preferred embodiment, the compound of Formula (I) may be selected from:
(azidomethylene)dibenzene;
1-(azido(phenyl)methyl)-4-chlorobenzene;
4,4′-(azidomethylene)bis(methoxybenzene);
(1-azidoethyl) benzene;
2-(1-azidoethyl) naphthalene;
(1-azidoethane-1, 1-diyl)dibenzene;
(azido methanetriyl)tribenzene;
5-(azidomethyl)benzo[d][1,3]dioxole;
5-(azidomethyl)-6-chlorobenzo[d][1,3]dioxole;
4-(azidomethyl)pyrene;
   a stereoisomer, a tautomer, a pharmaceutically acceptable salt or a pharmaceutically acceptable solvate thereof.

In an embodiment, the present disclosure provides a process of synthesizing organic azides by direct azidation of 3-hydroxy-2-oxindole compounds.

In an embodiment, the present disclosure provides a continuous flow process of synthesizing organic azides of formula (III), a stereoisomer, a tautomer, a pharmaceutically acceptable salt or a pharmaceutically acceptable solvate thereof, wherein the process comprises the step of reacting a compound of formula (IV) with trimethylsilyl azide and a catalyst Amberlyst-15 in a solvent;

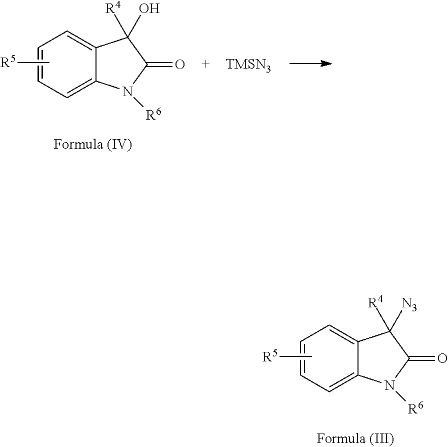

wherein R⁴, R⁵ and R⁶ are independently selected from one or more of H, halogen, —COOH, nitro, —NH₂, (C₁₋₆)alkoxy, substituted or unsubstituted (C₆₋₁₆)aryl, substituted or unsubstituted (C₁₋₆)alkyl; or substituted or unsubstituted —CH₂—(C₆₋₁₆)aryl; and
wherein the substituents may be selected from one or more of halogen, (C₁₋₆) alkyl, cyano, nitro, —NH₂, —COOH, (C₁₋₆)alkoxy, or combinations thereof.

In an embodiment, R⁴ may be selected from —CH₃; substituted or unsubstituted (C₆₋₁₆)aryl selected from phenyl; or substituted or unsubstituted —CH₂—(C₆₋₁₆)aryl selected from —CH₂—C₆H₅; wherein the substituents may be selected from one or more of halogen, —CH₃, or —OCH₃.

In an embodiment, R⁵ and R⁶ may be selected from one or more of H; —CH₃; halogen; or substituted or unsubstituted —CH₂—(C₆₋₁₆)aryl selected from —CH₂—C₆H₅.

In an embodiment, the ratio of Formula (IV) to TMSN₃ may be in the range of about 1:10 to about 10:1, preferably about 1:3.

In an embodiment, the present disclosure provides a compound of Formula (III), a stereoisomer, a tautomer, a pharmaceutically acceptable salt or a pharmaceutically acceptable solvate thereof obtained by the process as recited above.

In a preferred embodiment, the compounds of Formula (III) may be selected from:
3-azido-3-methyl indolin-2-one;
3-azido-3-phenyl indolin-2-one;
3-azido-3-(p-tolyl)indolin-2-one;
3-azido-3-(4-methoxyphenyl)indolin-2-one;
3-azido-3-benzyl indolin-2-one;
3-azido-3-(3,4-dimethoxybenzyl)indolin-2-one;
3-azido-3-(4-bromobenzyl)indolin-2-one;
3-azido-3-benzyl-6-chloro indolin-2-one;
3-azido-1,3-dibenzyl indolin-2-one;
3-azido-1,3-dimethyl indolin-2-one;
   a stereoisomer, a tautomer, a pharmaceutically acceptable salt or a pharmaceutically acceptable solvate thereof.

In an embodiment, the present disclosure provides a process of synthesizing organic 2-azido-2H-benzo[b][1,4]oxazin-3(4H)-one derivatives from peroxyoxyindoles.

In an embodiment, the present disclosure provides a continuous flow process of synthesizing organic azides of formula (V), a stereoisomer, a tautomer, a pharmaceutically acceptable salt or a pharmaceutically acceptable solvate thereof, wherein the process comprises the step of reacting a compound of formula (VI) with trimethylsilyl azide and a catalyst Amberlyst-15 in a solvent.

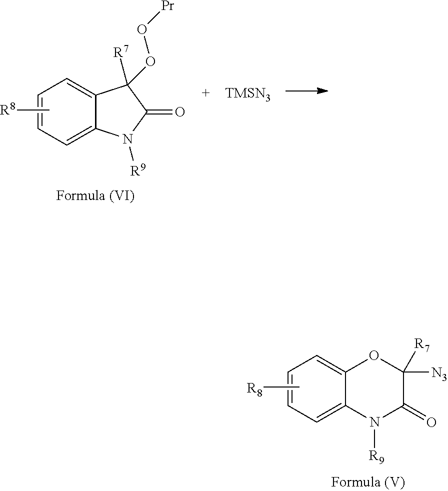

wherein R⁷ may be selected from H, (C₁₋₆)alkyl, substituted or unsubstituted (C₆₋₁₆)aryl; or substituted or unsubstituted —CH₂—(C₆₋₁₆)aryl;
wherein R⁸ and R⁹ may be independently selected from one or more of H, halogen, (C₁₋₆)alkyl, cyano, nitro, (C₁₋₆)alkoxy, substituted or unsubstituted —CH₂—(C₆₋₁₆)aryl, substituted or unsubstituted (C₆₋₁₆)aryl or combinations thereof;
wherein the substituent may be selected from one or more of halogen, (C₁₋₆)alkyl, cyano, nitro, (C₁₋₆)alkoxy, —COOH, —NH₂ or combinations thereof; and
wherein Pr is a protecting group.

The process with peroxides progresses with sequential skeletal rearrangement and azidation reaction. Without being bound to theory, it is believed that it progresses through deprotection of peroxo by the catalyst, followed by generation of N₃⁻ by the peroxo attack on TMS. The positive charged species undergoes ring expansion generating in situ carbocation which is attacked by N₃⁻ to give the compound.

In an embodiment, R⁷ may be selected from —CH₃; substituted or unsubstituted (C₆₋₁₆)aryl selected from phenyl; or substituted or unsubstituted —CH₂—(C₆₋₁₆)aryl selected from —CH₂—C₆H₅; wherein the substituents may be selected from —OCH₃, —CH₃ or halogen.

In an embodiment, R⁸ and R⁹ may be selected from H, halogen, —CH₃, or —CH₂—C₆H₅.

In an embodiment, the protecting group may be n-butyl, t-butyl, methyl, benzyl and the like.

In an embodiment, the present disclosure provides a compound of formula (V), a stereoisomer, a tautomer, a pharmaceutically acceptable salt or a pharmaceutically acceptable solvate thereof obtained by the process as recited above.

In a preferred embodiment, the compounds of formula (V) may be selected from:
2-azido-2-benzyl-2H-benzo[b][1,4]oxazin-3(4H)-one;
2-azido-2-methyl-2H-benzo[b][1,4]oxazin-3(4H)-one;
2-azido-2-(4-methoxyphenyl)-2H-benzo[b][1,4]oxazin-3(4H)-one;
2-azido-2-(2-fluorobenzyl)-2H-benzo[b][1,4]oxazin-3(4H)-one;
2-azido-2-(4-bromobenzyl)-2H-benzo[b][1,4]oxazin-3(4H)-one;
2-azido-2-benzyl-6-chloro-2H-benzo[b][1,4]oxazin-3(4H)-one;
2-azido-2-(4-bromobenzyl)-6-chloro-2H-benzo[b][1,4]oxazin-3(4H)-one;
2-azido-6-chloro-2-(4-methylbenzyl)-2H-benzo[b][1,4]oxazin-3(4H)-one;
2-azido-2,4-dimethyl-2H-benzo[b][1,4]oxazin-3(4H)-one;
2-azido-4-benzyl-2-methyl-2H-benzo[b][1,4]oxazin-3(4H)-one;
   a stereoisomer, a tautomer, a pharmaceutically acceptable salt or a pharmaceutically acceptable solvate thereof.

In an embodiment, the present disclosure provides a process of synthesizing organic azides by direct azidation of 9-alkyl/aryl-9H-fluoren-9-ol derivatives.

In an embodiment, the present disclosure provides a continuous flow process of synthesizing organic azides of formula (I′), a stereoisomer, a tautomer, a pharmaceutically acceptable salt or a pharmaceutically acceptable solvate thereof, wherein the process comprises the step of reacting a compound of Formula (II′) with trimethylsilyl azide and a catalyst Amberlyst-15 in a solvent;

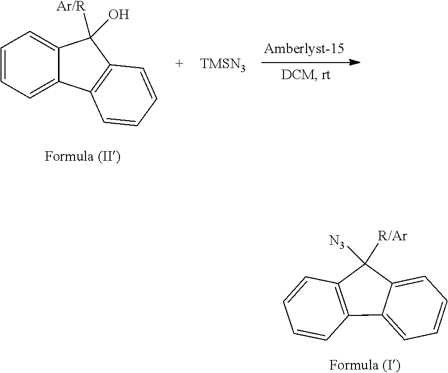

wherein Ar/R is selected from group consisting of substituted or unsubstituted (C₆₋₁₆)aryl, or substituted or unsubstituted (C₅₋₁₀)heterocycle substituted or unsubstituted (C₆₋₁₆)aryl, substituted or unsubstituted (C₁₋₆)alkyl, or substituted or unsubstituted —CH₂—(C₆₋₁₆)aryl; and one or more of halogen, (C₁₋₆)alkyl, cyano, nitro, —NH₂, (C₁₋₆)alkoxy, —COOH, or combinations thereof.

In a preferred embodiment, the compound of formula (I′) may be selected from:
9-azido-9-phenyl-9H-fluorene;
9-azido-9-(p-tolyl)-9H-fluorene;
9-azido-9-(4-methoxyphenyl)-9H-fluorene;
9-([1,1′-biphenyl]-4-yl)-9-azido-9H-fluorene;
9-azido-9-hexyl-9H-fluorene;
9-azido-2,7-dibromo-9-(4-methoxyphenyl)-9H-fluorene;
9-azido-9-benzyl-9H-fluorene;
9-azido-9-(3-phenoxybenzyl)-9H-fluorene;
9-([1,1′-biphenyl]-4-ylmethyl)-9-azido-9H-fluorene;
9-azido-9-(4-methoxybenzyl)-9H-fluorene;
9,9′-diazido-9H,9′H-9,9′-bifluorene;
9-azido-2-bromo-9-phenyl-9H-fluorene,
9-azido-2,7-dibromo-9-phenyl-9H-fluorene;
9-azido-2,7-dibromo-9-(p-tolyl)-9H-fluorene; or
   a stereoisomer, a tautomer, a pharmaceutically acceptable salt or a pharmaceutically acceptable solvate thereof.

In an embodiment, the ratio of compound (VI) to TMSN₃ may be in the range of about 1:10 to about 10:1, preferably about 1:3.

In an embodiment, the process may be performed in the presence of a solvent selected from dichloromethane, 1,2-dichloroethane, and the like; preferably the solvent is dichloromethane. The catalyst may be packed in a column and the residence time for the reaction may be at least 5 minutes, at least 10 minutes, at least 20 minutes, at least 30 minutes, at least 60 minutes, at least 120 minutes or at least 180 minutes, preferably about 21 minutes.

In an embodiment, the process may be performed at a temperature range of about 15° C. to about 100° C., preferably about 25° C. to 80° C.

In an embodiment, Amberlyst-15 may be present in a ratio of about 1:1 w/w to about 1:20 w/w to the reactant compound, preferably about 1:1 w/w of the reactant compound. In an embodiment, the flow rate of the reactant in the continuous flow process may be about 0.08 mL/min to about 0.5 mL/min, preferably about 0.1 mL/min. In an embodiment, the process may be carried out under a pressure of about 1 to 3 bar, preferably about 0-1 bar.

The process tolerates both electron withdrawing as well as electron donating groups to afford the respective azides via direct nucleophilic substitution reaction. It also provides azides for more sterically hindered alcohols.

The process efficiently synthesizes azides including those with a quaternary stereocenter.

Continuous flow reactors provide an ideal tool for synthesis of potentially explosive organic compounds such as the azides of the present disclosure due to its intrinsically small volume leading to very effective collision and highly controlled reaction conditions. The advent of continuous flow as a green tool manifests enhanced heat and mass transfer, precise residence time control, shorter process times, increased safety, reproducibility, better product quality and easy scalability. The process decreases waste generation from the two-step process conventionally employed for azide synthesis.

Amberlyst-15 can serve as an excellent source of acid and also can be recovered and reused several times.

The process of the present disclosure increases implementation of the azide synthesis not just in academia but also into the fine chemical manufacturing sector. Thus, the process aids in the scale up of the synthesis mitigating the safety concerns of the explosive and high energy molecules which decompose with heat, light, or shock under batch conditions. The yields generated are also higher for the continuous flow method than the batch conditions.

The process of the present disclosure may provide high yields of at least 10%, at least 20%, at least 30%, at least 40%, at least 50%, at least 60%, at least 70%, at least 80%, at least 85%, at least 90%, at least 95%, or at least 99%.

The azides obtained from the above processes can be further used to synthesize crucial chemical compounds with industrial applicability.

Application of the azides may be demonstrated towards a Staudinger reduction to generate amines.

In an embodiment, the present disclosure provides a process of synthesizing amine derivatives by reduction of the synthesized azides in the presence of PPh₃.

In an embodiment, the compound of formula (I) undergoes reduction with triphenylphosphine in tetrahydrofuran and water to give a compound of formula (VII).

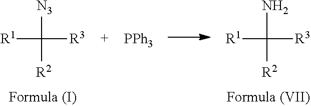

In an embodiment, the compound of formula (III) undergoes reduction with triphenylphosphine in tetrahydrofuran and water to give a compound of formula (VIII).

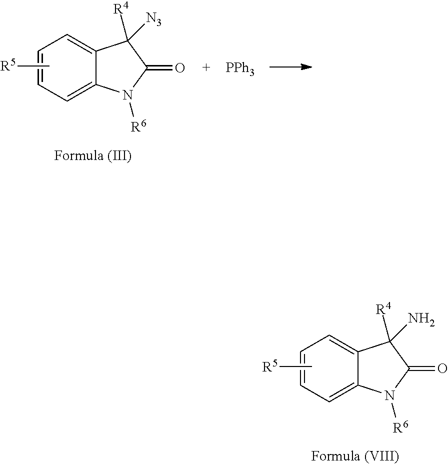

In an embodiment, the compound of formula (V) undergoes reduction with triphenylphosphine in tetrahydrofuran and water to give a compound of formula (IX).

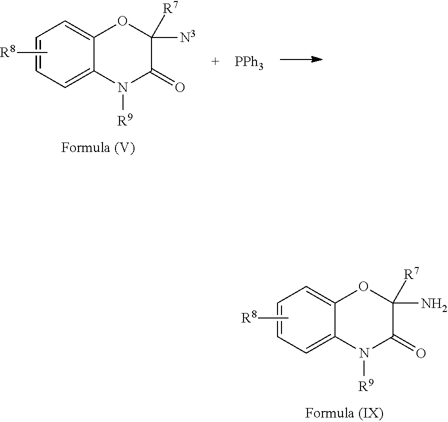

Application of the azide may be demonstrated towards a click reaction with alkyne to generate a quaternary stereocenter encompassing a triazole moiety.

In an embodiment, the present disclosure provides a continuous flow process of synthesizing triazole functionalized derivatives from azides using a Cu-catalyst.

In an embodiment, the compound of formula (I) undergoes a reaction with an alkyne of formula (X) in a suitable solvent and a Cu-catalyst to give a compound of formula (XI); wherein R¹⁰ is a C₁₋₆ alkyl or C₆₋₁₀aryl.

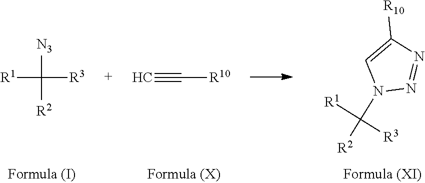

In an embodiment, the compound of formula (III) undergoes a reaction with an alkyne of formula (X) in a suitable solvent and a Cu-catalyst to give a compound of formula (XII); wherein R¹⁰ is a C₁₋₆ alkyl or C₆₋₁₀aryl.

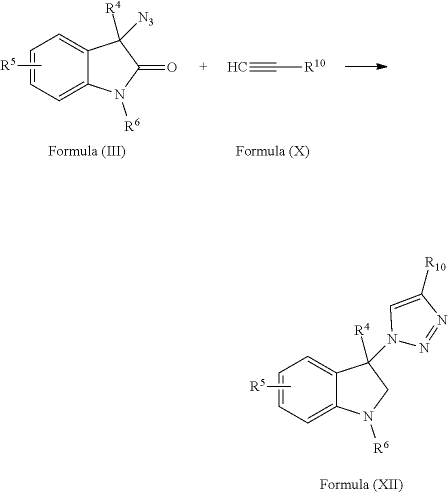

In an embodiment, the compound of formula (V) undergoes a reaction in batch mode with an alkyne of formula (X) in a suitable solvent and a Cu-catalyst to give a compound of formula (XIII); wherein R¹⁰ is a C₁₋₆ alkyl or C₆₋₁₀aryl.

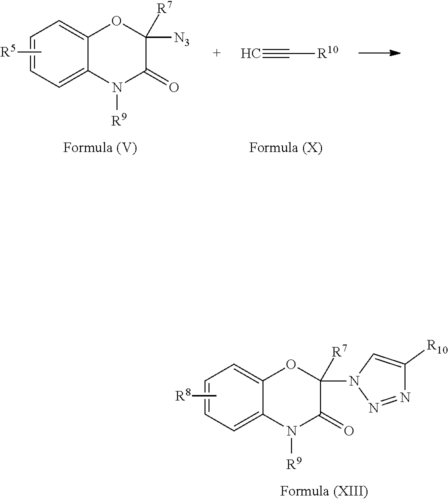

The above process may be carried out in batch mode.

In an embodiment, the compound of Formula X is ethynyl benzene. The suitable solvent may be a mixture of tertiary butyl alcohol and water.

Application of the azide may be demonstrated towards rearrangement of the azide to generate quinoxalinone derivatives.

In an embodiment, the present disclosure provides a continuous flow process of synthesizing quinoxaline-2(1H)-one derivatives from azides in a reagent-less condition.

In an embodiment, the compound of Formula (III) may be heated at about 100° C. to about 200° C., preferably about 180° C., to give a compound of Formula (XIV); wherein the process is a continuous flow process.

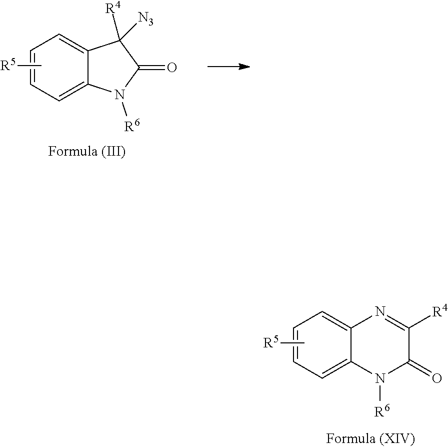

In an embodiment, the pressure may be in the range of about 1 to 3 bars.

While the foregoing describes various embodiments of the disclosure, other and further embodiments of the disclosure may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

### EXAMPLES

The present invention is further explained in the form of the following examples. However, it is to be understood that the following examples are merely illustrative and are not to be taken as limitations upon the scope of the invention.

### Materials and Methods

All the chemicals were purchased from Sigma-Aldrich and SD Fine Chemicals and were used without further modification. All solvents were purchased from Rankem and Finar Chemicals. Deuterated solvents were used as received. Column chromatographic separations were performed over 100-200 silica-gel. Visualization was accomplished with UV light. The flow chemistry experiments were carried out on Vapourtec R-series with glass columns (Omni fit, 6.6×150 mm) and Vapourtec R-series with SS coil reactor (10 ml). The ¹H and ¹³C {¹H} NMR spectra were recorded on 400 and 100 MHz, respectively, using Bruker or JEOL spectrometers. HRMS spectra were obtained with Waters-synapt G2 using electrospray ionization (ESI-TOF). Infrared (ATIR) spectra were obtained with a Bruker Alpha-E infrared spectrometer. Single-crystal diffraction analysis data were collected at 100K with a BRUKER KAPPA APEX III CCD Duo diffractometer (operated at 1500 W power: 50 kV, 30 mA) using graphite monochromatic Mo Kα radiation and Cu-Kα radiation.

Abbreviations used in the NMR follow-up experiments: b, broad; s, singlet; d, doublet; t, triplet; q, quartet; td, dd doublet of triplet and double doublet; m, multiplet, tt, triplet of triplets and ddd, doublet of doublet of doublets.

### Example 1: Continuous Flow Synthesis of Compounds of Formula (I) and (III)

Reaction was conducted under continuous flow for the compound 1a as per Scheme I. In a typical procedure, 0.1M solution of diphenylmethanol(1a) in dichloromethane and 3 equivalents of 0.3M azidotrimethylsilane(2a) was premixed and flown through Omnifit® (6.6×150 mm) packed bed column (Vaportec R-series) packed with Amberlyst-15 up to 5 cm (1.0 g, swollen up to 6 cm after passing solvent) of bed at room temperature at 0-1 bar pressure with 0.1 mL/min flow rate and residence time of 21 minutes. After reaction completion, the catalyst bed was washed with dichloromethane. Volatile component was evaporated using vacuum. The residue was directly purified by silica gel chromatography (EtOAc:hexane=1:99 to 5:95). Amberlyst-15 bed was recycled by washing with DCM and reused for the other substrates.

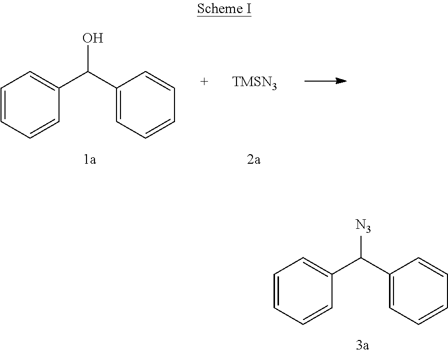 [SMILES: OC(c1ccccc1)c1ccccc1 ; "TMSN3" ; [N-]=[N+]=NC(c1ccccc1)c1ccccc1]

Compounds of the Table 1 were synthesized using the same procedure as recited above, except that compound 31 resulted in 93% yield when heated at 80° C. in DCE solvent.

TABLE 1
Compounds synthesized by continuous flow process

| Name | Structure | % yield* | Characterization data |
|---|---|---|---|
| 3a | 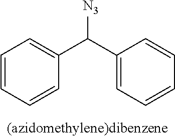 [SMILES: [N-]=[N+]=NC(c1ccccc1)c1ccccc1] | 99 | Colorless oil; 6144 mg ¹H NMR (400 MHz, CDCl₃) δ [ppm] = 7.40 (m, 10H), 5.79 (s, 1H). ¹³C NMR (100 MHz, CDCl₃) δ [ppm] = 139.7, 128.8, 128.1, 127.5, 68.6. IR (neat): 2096, 1455, 1238 cm⁻¹. HRMS (ESI-TOF) m/z: [M + H]⁺ calcd. for C₁₃H₁₂N: 182.0970; found: 182.0966 |
| 3b | 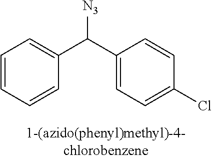 [SMILES: [N-]=[N+]=NC(c1ccccc1)c1ccc(Cl)cc1] | 97 | Pale yellow oil; 117.8 mg ¹H NMR (400 MHz, CDCl₃) δ [ppm] = 7.26 (m, 9H), 5.63 (s, 1H). ¹³C NMR (100 MHz, CDCl3) δ [ppm] = 139.2, 138.3, 134.0, 128.9, 128.4, 127.5, 67.9. IR (neat): 2098, 1659, 1495, 1087, 703 cm⁻¹. HRMS (ESI-TOF) m/z: [M + H − N₂]⁺ calcd for C₁₃H₁₁ClN: 216.0580; found: 216.0571 |
| 3c | 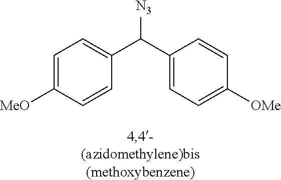 [SMILES: COc1ccc(C(N=[N+]=[N-])c2ccc(OC)cc2)cc1] | 80 | pale yellow; 107.6 mg ¹H NMR (400 MHz, CDCl₃) δ [ppm] = 7.27 (dq, J = 6.7, 2.4 Hz, 4H), 6.93 (m, 4H), 5.69 (s, 1H), 3.84 (s, 6H). ¹³C NMR (100 MHz, CDCl₃) δ [ppm] = 159.3, 132.1, 128.7, 114.1, 67.7, 55.3. IR (neat): 2092, 1611, 1508, 1243 cm⁻¹ HRMS (ESI-TOF) m/z: [M + H − N₂]⁺ calcd for C₁₅H₁₆NO₂: 242.1181; found: 242.1174 |
| 3d | 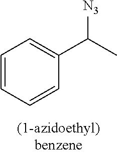 | 94 | Colorless oil; 138.0 mg ¹H NMR (400 MHz, CDCl₃) δ [ppm] = 7.37 (m, 5H), 4.63 (m, 1H), 1.54 (d, J = 6.7 Hz, 3H). ¹³C NMR (100 MHz, CDCl₃) δ [ppm] = 141.0, 128.9, 128.5, 126.5, 61.2, 21.7. IR (neat): 2099, 1246 cm⁻¹ |
| 3e | 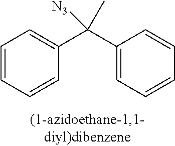 | 91 | Pale yellow oil; 203.0 mg ¹H NMR (400 MHz, CDCl₃) δ [ppm] = 7.35 (m, 10H), 2.07 (s, 3H). ¹³C NMR (100 MHz, CDCl3) δ [ppm] = 144.3, 128.5, 127.6, 126.7, 69.5, 27.5. IR (neat): 2087, 1492, 1444, 1238 cm⁻¹ HRMS (ESI-TOF) m/z: [M + H − N₂]⁺ calcd for C₁₄H₁₄N: 196.1126; found: 196.1129 |
| 3f | 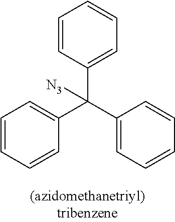 [SMILES: [N-]=[N+]=NC(c1ccccc1)(c1ccccc1)c1ccccc1] | 86 | White solid; 122.5 mg ¹H NMR (400 MHz, CDCl₃) δ [ppm] = 7.32 (m, 15H). ¹³C NMR (100 MHz, CDCl₃) δ [ppm] = 143.3, 128.6, 128.3, 127.8. IR (neat): 2096, 1455, 1238 cm⁻¹ HRMS (ESI-TOF) m/z: [M + H − N₂]⁺ calcd for C₁₉H₁₆N: 258.1283; found: 258.1290 |
| 3g | 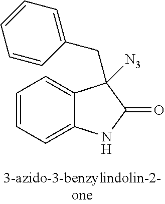 [SMILES: [N-]=[N+]=NC1(Cc2ccccc2)C(=O)Nc2ccccc21] | 93 | Pale yellow semi-solid; 123.0 mg ¹H NMR (400 MHz, CDCl₃) δ [ppm] = 8.36 (s, 1H), 7.26 (m, 1H), 7.15 (m, 3H), 7.06 (m, 2H), 7.00 (m, 2H), 6.80 (d, J = 7.7 Hz, 1H), 3.36 (d, J = 13.1 Hz, 1H), 3.24 (d, J = 13.2 Hz, 1H). ¹³C NMR (100 MHz, CDCl₃) δ [ppm] = 176.5, 140.5, 133.2, 130.5, 130.3, 128.1, 127.4, 126.5, 125.2, 123.0, 110.6, 68.0, 41.6. IR (neat): 2101, 1719, 1472 cm⁻¹ HRMS (ESI-TOF) m/z: [M + Na]⁺ calcd for C₁₅H₁₂N₄ONa: 287.0909; found: 287.0909 |
| 3h | 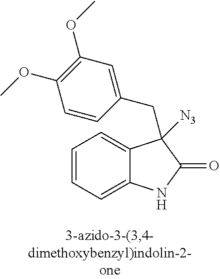 [SMILES: COc1ccc(CC2(N=[N+]=[N-])C(=O)Nc3ccccc32)cc1OC] | 60 | Pale yellow semi-solid; 98.0 mg ¹H NMR (400 MHz, CDCl₃) δ [ppm] = 8.65 (s, 1H), 7.24 (m, 1H), 7.14 (d, J = 7.1 Hz, 1H), 7.06 (m, 1H), 6.79 (d, J = 7.7 Hz, 1H), 6.59 (m, 2H), 6.39 (d, J = 2.0 Hz, 1H), 3.76 (s, 3H), 3.60 (s, 3H), 3.29 (d, J = 13.3 Hz, 1H), 3.19 (d, J = 13.1 Hz, 1H) ¹³C NMR (100 MHz, CDCl₃) δ [ppm] = 176.4, 148.2, 140.9, 130.3, 126.8, 125.5, 125.1, 122.9, 122.8, 113.4, 110.8, 110.7, 68.1, 55.7, 55.6, 41.3. IR (neat): 2101, 1635 cm⁻¹ HRMS (ESI-TOF) m/z: [M + Na]⁺ calcd for C₁₇H₁₆N₄O₃Na: 347.1120; found: 347.1118. |
| 3i | 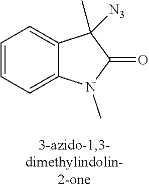 | 28 | Colourless oil; 28.3 mg ¹H NMR (400 MHz, CDCl₃) δ [ppm] = 7.35 (m, 2H), 7.12 (td, J = 7.6, 0.7 Hz, 1H), 6.87 (d, J = 7.8 Hz, 1H), 3.22 (s, 3H), 1.67 (s, 3H). ¹³C NMR (100 MHz, CDCl₃) δ [ppm] = 175.0, 142.9, 130.2, 128.9, 123.5, 108.9, 63.3, 26.5, 21.5. IR (neat): 2098, 1720, 1616, 1471 cm⁻¹ HRMS (ESI-TOF) m/z: [M + H − N₂]⁺ calcd for C₁₀H₁₁N₂O: 175.0871; found: 175.0862 |
| 3j | 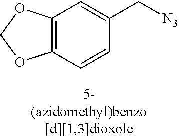 [SMILES: [N-]=[N+]=NCc1ccc2c(c1)OCO2] | 91 | Colourless oil; 161 mg ¹H NMR (400 MHz, CDCl₃) δ [ppm] = 6.78 (m, 3H), 5.97 (s, 2H), 4.23 (s, 2H). ¹³C NMR (100 MHz, CDCl₃) δ [ppm] = 148.1, 147.8, 129.1, 122.0, 108.8, 108.4, 101.3, 54.8. IR (neat): 2091, 1488, 1443 cm⁻¹. |
| 3k | 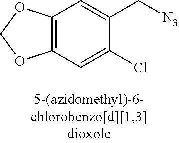 [SMILES: [N-]=[N+]=NCc1cc2c(cc1Cl)OCO2] | 97 | Colourless oil; 103 mg ¹H NMR (400 MHz, CDCl₃) δ [ppm] = 6.87 (s, 1H), 6.83 (s, 1H), 5.99 (s, 2H), 4.37 (s, 2H). ¹³C NMR (100 MHz, CDCl₃) δ [ppm] = 148.3, 147.0, 126.3, 126.0, 110.1, 109.7, 102.1, 52.2. IR (neat): 2098, 1505, 1476, 1235 cm⁻¹ HRMS (ESI-TOF) m/z: [M + H − N₂]⁺ calcd for C₈H₇NO₂Cl: |
| | | | 184.0165; found: 184.0163. |
| 3l | 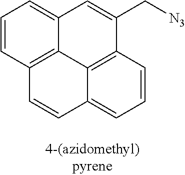 [SMILES: [N-]=[N+]=NCc1cc2cccc3ccc4cccc1c4c32] | 80 | Pale yellow solid; 103.0 mg ¹H NMR (400 MHz, CDCl₃) δ [ppm] = 8.09 (m, 9H), 4.99 (s, 2H). ¹³C NMR (100 MHz, CDCl₃) δ [ppm] = 131.8, 131.3, 130.8, 129.3, 128.4, 128.3, 127.9, 127.5, 127.4, 126.3, 125.7, 125.6, 125.1, 124.7, 122.7, 53.2. IR (neat): 2031, 1508, 1291, 841 cm⁻¹ HRMS (ESI-TOF) m/z: [M + H − N₂]⁺ calcd for C₁₇H₁₂N: 230.0970; found: 230.0970 |

*Mentioned yields are isolated yields

Some other compounds that can be synthesized in continuous flow method are provided in Table 2 below.

TABLE 2
Compounds of Formula I and III

| Name | Structure | Characterization data |
|---|---|---|
| 3m | 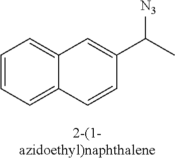 | Colorless oil; ¹H NMR (400 MHz, CDCl₃) δ [ppm] = 8.16 (dd, J = 8.2, 3.4 Hz, 1H), 7.92 (ddd, J = 11.2, 8.0, 2.7 Hz, 2H), 7.59 (m, 4H), 5.40 (m, 1H), 1.78 (dd, J = 6.8, 2.8 Hz, 3H). ¹³C NMR (100 MHz, CDCl3) δ [ppm] = 136.2, 134.0, 130.6, 129.1, 128.8, 126.5, 125.9, 125.4, 123.6, 123.1, 57.6, 20.7 IR (neat): 2009, 1508, 1241 cm⁻¹ HRMS (ESI- TOF) m/z: [M + H − N₂]⁺ calcd for C₁₂H₁₂N: 170.0970; found: 170.0968 |
| 3n | 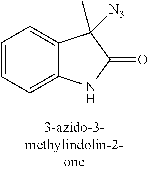 | Yellow solid; ¹H NMR (400 MHz, CDCl₃) δ [ppm] = 8.69 (s, 1H), 7.31 (t, J = 8.2 Hz, 2H), 7.11 (t, J = 7.6 Hz, 1H), 6.96 (d, J = 7.7 Hz, 1H), 1.70 (s, 3H). ¹³C NMR (100 MHz, CDCl₃) δ [ppm] = 177.4, 140.1, 130.3, 129.3, 124.0, 123.5, 110.8, 63.9, 21.6. IR (neat): 2089, 1716, 1620, 1472, 1201 cm⁻¹ HRMS (ESI-TOF) m/z: [M + H − N₂]⁺ calcd for C₉H₉N₂O: 161.0715; found: 161.0709 |
| 3o | 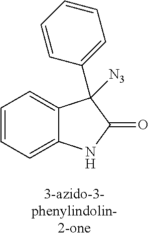 [SMILES: [N-]=[N+]=NC1(c2ccccc2)C(=O)Nc2ccccc21] | Pale yellow solid; ¹H NMR (400 MHz, CDCl₃) δ [ppm] = 8.77 (s, 1H), 7.39 (m, 6H), 7.26 (m, 1H), 7.12 (td, J = 7.6, 1H), 7.00 (d, J = 7.8 Hz, 1H). ¹³C NMR (100 MHz, CDCl₃) δ [ppm] = 176.0, 140.7, 136.3, 130.6, 129.1, 129.0, 128.9, 126.6, 125.6, 123.8, 111.1, 70.4. IR (neat): 3249, 2101, 1730, 1717, 1622, 1476 cm⁻¹ HRMS (ESI-TOF) m/z: [M + Na]⁺ calcd for C₁₄H₁₀N₄ONa: 273.0752; found: 273.0759 |
| 3p | 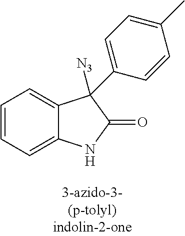 [SMILES: Cc1ccc(C2(N=[N+]=[N-])C(=O)Nc3ccccc32)cc1] | Pale yellow solid; ¹H NMR (400 MHz, CDCl₃) δ [ppm] = 9.77 (s, 1H), 7.38 (m, 3H), 7.31 (d, J = 7.5 Hz, 1H), 7.25 (d, J = 8.1 Hz, 2H), 7.16 (td, J = 7.6, 0.8 Hz, 1H), 7.03 (d, J = 7.8 Hz, 1H), 2.39 (s, 3H). ¹³C NMR (100 MHz, CDCl₃) δ [ppm] = 176.9, 140.9, 138.9, 133.2, 130.5, 129.8, 129.1, 126.5, 125.3, 123.6, 111.4, 70.5, 21.2. IR (neat): 2098, 1725, 1619, 1471 cm⁻¹ HRMS (ESI-TOF) m/z: [M + Na]⁺ calcd for C₁₅H₁₂N₄ONa: 287.0909; found: 287.0908 |
| 3q | 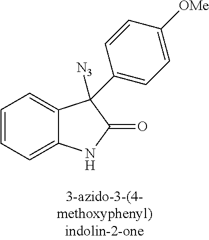 [SMILES: COc1ccc(C2(N=[N+]=[N-])C(=O)Nc3ccccc32)cc1] | Pale yellow liquid; ¹H NMR (400 MHz, CDCl₃) δ [ppm] = 9.46 (s, 1H), 7.31 (m, 2H), 7.23 (m, 2H), 7.04 (td, J = 7.6, 0.8 Hz, 1H), 6.91 (d, J = 7.8 Hz, 1H), 6.83 (m, 2H), 3.71 (s, 3H). ¹³C NMR (100 MHz, CDCl₃) δ [ppm] = 176.7, 160.1, 140.9, 130.5, 128.9, 128.1, 125.4, 123.6, 114.4, 111.3, 70.2, 55.4. IR (neat): 2102, 1725, 1619, 1510 cm⁻¹ HRMS (ESI-TOF) m/z: [M + Na]⁺ calcd for C₁₅H₁₂N₄O₂Na: 303.0858; found: 303.0851 |
| 3r | 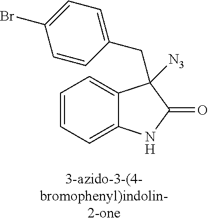 [SMILES: [N-]=[N+]=NC1(Cc2ccc(Br)cc2)C(=O)Nc2ccccc21] | Yellow solid; ¹H NMR (400 MHz, CDCl₃) δ [ppm] = 7.93 (s, 1H), 7.27 (m, 3H), 7.09 (m, 2H), 6.86 (m, 2H), 6.79 (d, J = 7.8 Hz, 1H), 3.29 (d, J = 13.1 Hz, 1H), 3.21 (d, J = 13.1 Hz, 1H). ¹³C NMR (100 MHz, CDCl₃) δ [ppm] = 175.6, 140.4, 132.2, 131.4, 130.5, 126.3, 125.1, 123.2, 121.7, 110.7, 67.7, 41.1. IR (neat): 2104, 1652 cm⁻¹ |
| 3s | 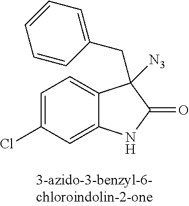 [SMILES: [N-]=[N+]=NC1(Cc2ccccc2)C(=O)Nc2cc(Cl)ccc21] | Yellow white semi-solid; ¹H NMR (400 MHz, CDCl₃) δ [ppm] = 8.19 (s, 1H), 7.19 (m, 3H), 6.99 (m, 4H), 6.82 (t, J = 3.5 Hz, 1H), 3.35 (d, J = 13.2 Hz, 1H), 3.22 (d, J = 13.2 Hz, 1H). ¹³C NMR (100 MHz, CDCl₃) δ [ppm] = 176.1, 141.6, 136.1, 132.9, 130.6, 128.4, 127.7, 126.3, 125.0, 123.1, 111.4, 67.5, 41.5. IR (neat): 2114, 1725, 1614 cm⁻¹ HRMS (ESI- TOF) m/z: [M + Na]⁺ calcd for C₁₅H₁₁N₄OClNa: 321.0519; found: 321.0514 |
| 3t | 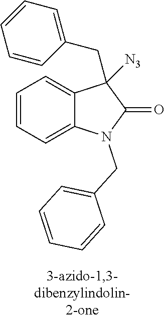 [SMILES: [N-]=[N+]=NC1(Cc2ccccc2)C(=O)N(Cc2ccccc2)c2ccccc21] | Yellow white solid; ¹H NMR (400 MHz, CDCl₃) δ [ppm] = 7.86 (dd, J = 8.3, 1.3 Hz, 1H), 7.49 (d, J = 7.5 Hz, 2H), 7.39 (m, 1H), 7.30 (m, 5H), 7.22 (m, 5H), 5.46 (s, 2H), 4.33 (s, 2H). ¹³C NMR (100 MHz, CDCl₃) δ [ppm] = 159.6, 155.0, 137.2, 135.4, 133.2, 132.8, 130.1, 130.0, 129.7, 129.0, 128.6, 127.8, 127.0, 126.7, 123.8, 114.5, 46.1, 40.9. IR (neat): 2101, 1720, 1614, 1468 cm⁻¹ HRMS (ESITOF) m/z: [M + Na]⁺ calcd for C₂₂H₁₈N₄ONa: 377.1378; found: 377.138 |

### Example 2

### Continuous Flow Synthesis of Compounds of Formula (V)

For synthesizing 2-azido-2-benzyl-2H-benzo[b][1,4]oxazin-3(4H)-one (5a) from peroxide 4a the Scheme 2 was used. 0.1M solution of peroxide compound of 4a in dichloromethane and 3 equivalents of azidotrimethylsilane (2a, 0.3M) was premixed and flown through Omnifit® (6.6×150 mm) packed bed column packed with Amberlyst-15 up to 5 cm (1.0 g, swollen up to 6 cm after passing solvent) of bed at room temperature at 0-1 bar pressure with 0.1 mL/min flow rate and residence time of 21 min. After reaction completion, the catalyst bed was washed with dichloromethane. Volatile component was evaporated using vacuum. The residue was directly purified by silica gel chromatography (EtOAc:hexane=10:90).

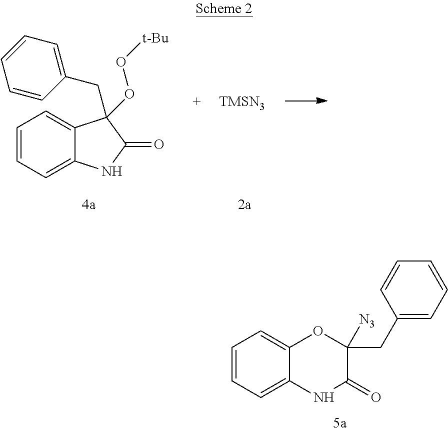

Compounds of Table 3 were prepared using the same scheme as shown above.

TABLE 3
Compounds synthesized by continuous flow process

| Name | Structure | % yield* | Characterization data |
|---|---|---|---|
| 5a | 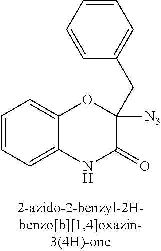 [SMILES: [N-]=[N+]=NC1(Cc2ccccc2)Oc2ccccc2NC1=O] | 68 | Pale yellow solid; 95.0 mg ¹H NMR (400 MHz, CDCl₃) δ [ppm] = 9.92 (s, 1H), 7.46 (m, 2H), 7.32 (m, 3H), 7.08 (m, 3H), 6.93 (m, 1H), 3.69 (d, J = 14.0 Hz, 1H), 3.50 (d, J = 14.0 Hz, 1H). ¹³C NMR (100 MHz, CDCl₃) δ [ppm] = 162.5, 140.8, 133.1, 131.3, 128.3, 127.6, 125.6, 124.8, 123.9, 117.8, 115.9, 91.6, 40.4. IR (neat): 2111, 1607, 1501, 1210 cm⁻¹ HRMS (ESI-TOF) m/z: [M + Na]⁺ calcd for C₁₅H₁₂N₄O₂Na: 303.0858; found: 303.0864. |
| 5b | 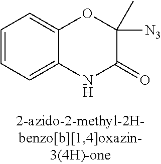 | 94 | Pale yellow solid; 96.0 mg ¹H NMR (400 CDCl₃) δ [ppm] = 9.29 (s, 1H), 7.06 (m, 3H), 6.93 (dd, J = 4.5, 2.4 Hz, 1H), 1.98 (s, 3H). ¹³C NMR (100 MHz, CDCl₃) δ [ppm] = 163.0, 141.0, 126.1, 124.7, 123.9, 117.8, 116.0, 90.3, 20.7. IR (neat): 31 2118, 1699, 1506 cm⁻¹. HRMS (ESI-TOF) m/z: [M + H − N₂]⁺ calcd for C₉H₉N₂O₂: 177.0664; found: 177.0668. |
| 5c | 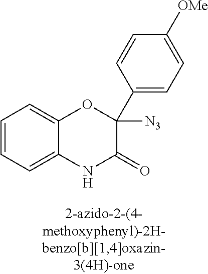 [SMILES: COc1ccc(C2(N=[N+]=[N-])Oc3ccccc3NC2=O)cc1] | 72 | Yellow semi-solid; 57.0 mg ¹H NMR (400 MHz, CDCl₃) δ [ppm] = 8.26 (d, J = 8.2 Hz, 1H), 7.62 (m, 2H), 7.38 (d, J = 7.9 Hz, 1H), 7.20 (d, J = 7.8 Hz, 1H), 6.77 (s, 4H), 3.76 (s, 3H). ¹³C NMR (100 MHz, CDCl₃) δ [ppm] = 154.5, 153.8, 149.7, 139.7, 134.5, 132.7, 124.7, 120.6, 116.1, 114.9, 102.2, 55.9. IR (neat): 2151, 1720, 1510, 1222 cm⁻¹ HRMS (ESI-TOF) m/z: [M + H − N₂]⁺ calcd for C₁₅H₁₃N₂O₃: 269.0926; found: 269.0934 |
| 5d | 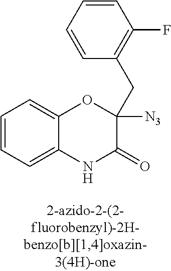 [SMILES: [N-]=[N+]=NC1(Cc2ccccc2F)Oc2ccccc2NC1=O] | 51 | Pale yellow solid; 20.7 mg ¹H NMR (400 MHz, CDCl₃) δ [ppm] = 9.52 (s, 1H), 7.47 (m, 3H), 7.26 (m, 1H), 7.06 (m, 5H), 6.91 (m, 1H), 3.68 (d, J = 14.3 Hz, 1H), 3.61 (d, J = 14.4 Hz, 1H). ¹³C NMR (100 MHz, CDCl₃) δ [ppm] = 163.3, 162.5, 160.9, 140.7, 132.9 (d, J = 3.5 Hz), 129.6 (d, J = 8.2 Hz), 125.5, 124.8, 123.99 (d, J = 5.4 Hz), 120.5 (d, J = 15.2 Hz), 118.0, 116.0, 115.6, 115.4, 91.5, 33.1 (d, J = 2.5 Hz). IR (neat): 2110, 1690, 1501, 750 cm⁻¹ HRMS (ESI-TOF) m/z: [M + H − N₂]⁺ calcd for C₁₅H₁₂N₂O₂F: 271.0883; found: 271.0890. |
| 5e | 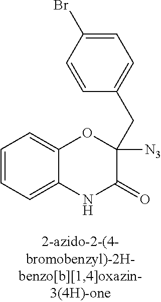 [SMILES: [N-]=[N+]=NC1(Cc2ccc(Br)cc2)Oc2ccccc2NC1=O] | 66 | Pale yellow solid; 70.8 mg ¹H NMR (400 MHz, CDCl₃) δ [ppm] = 9.70 (s, 1H), 7.41 (d, J = 8.4 Hz, 2H), 7.31 (d, J = 8.4 Hz, 2H), 7.07 (m, 3H), 6.90 (m, 1H), 3.64 (d, J = 14.0 Hz, 1H), 3.41 (d, J = 14.0 Hz, 1H). ¹³C NMR (100 MHz, CDCl3) δ [ppm] = 162.4, 140.6, 133.0, 132.1, 131.5, 125.4, 124.9, 124.0, 121.9, 117.8, 116.0, 91.4, 39.8. IR (neat): 2113, 1698, 1504, 751 cm⁻¹ HRMS (ESI-TOF) m/z: [M + H − N2]+ calcd for C₁₅H₁₂BrN₂O₂: 331.0082; found: 331.0081 |
| 5f | 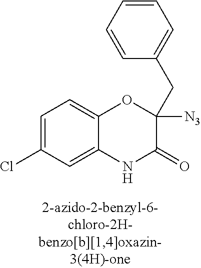 [SMILES: [N-]=[N+]=NC1(Cc2ccccc2)Oc2ccc(Cl)cc2NC1=O] | 40 | White solid; 32.0 mg ¹H NMR (400 MHz, CDCl₃) δ [ppm] = 8.45 (s, 1H), 7.34 (m, 5H), 6.99 (m, 2H), 6.82 (d, J = 1.7 Hz, 1H), 3.66 (d, J = 13.9 Hz, 1H), 3.45 (d, J = 13.9 Hz, 1H). ¹³C NMR (100 MHz, CDCl3) δ [ppm] = 161.7, 139.4, 132.8, 131.3, 128.9, 128.4, 127.7, 126.6, 124.5, 119.0, 115.6, 91.6, 40.3. IR (neat): 2114, 1699 cm⁻¹ HRMS (ESI-TOF) m/z: [M + H − N₂]⁺ calcd for C₁₅H₁₂ClN₂O₂: 287.0587; found: 287.0581. |
| 5g | 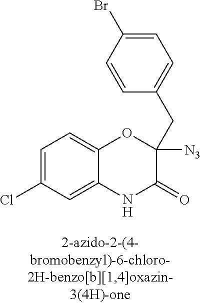 [SMILES: [N-]=[N+]=NC1(Cc2ccc(Br)cc2)Oc2ccc(Cl)cc2NC1=O] | 28 | White semi-solid; 27.6 mg ¹H NMR (400 MHz, CDCl₃) δ [ppm] = 9.28 (s, 1H), 7.42 (d, J = 8.4 Hz, 2H), 7.28 (d, J = 8.4 Hz, 2H), 7.02 (m, 2H), 6.87 (s, 1H), 3.62 (d, J = 13.9 Hz, 1H), 3.40 (d, J = 14.0 Hz, 1H). ¹³C NMR (100 MHz, CDCl₃) δ [ppm] = 162.0, 139.2, 133.0, 131.8, 131.6, 129.1, 126.4, 124.7, 122.0, 119.0, 115.9, 91.3, 39.7. IR (neat): 2111, 1704 cm⁻¹ HRMS (ESI-TOF) m/z: [M + H − N₂]⁺ calcd for C₁₅H₁₁N₂O₂BrCl: 364.9692; found: 364.9694 |
| 5h | 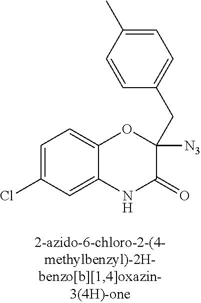 | 45 | Pale yellow solid; 26.2 mg ¹H NMR (400 MHz, CDCl₃) δ [ppm] = 9.66 (s, 1H), 7.30 (d, J = 7.8 Hz, 2H), 7.11 (d, J = 7.7 Hz, 2H), 7.01 (s, 2H), 6.89 (s, 1H), 3.62 (d, J = 14.0 Hz, 1H), 3.43 (d, J = 14.1 Hz, 1H), 2.31 (s, 3H). ¹³C NMR (100 MHz, CDCl₃) δ [ppm] = 162.7, 139.5, 137.4, 131.1, 129.7, 129.1, 128.9, 126.6, 124.5, 118.9, 115.9, 91.6, 39.9, 21.2. IR (neat): 2117, 1698, 1645 cm⁻¹ HRMS (ESI-TOF) m/z: [M + H − N₂]⁺ calcd for C₁₆H₁₄N₂O₂Cl: 301.0744; found: 301.0738 |
| 5i | 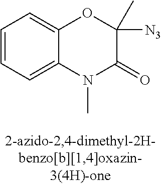 | 65 | White solid; 85.0 mg ¹H NMR (400 MHz, CDCl₃) δ [ppm] = 7.12 (dq, J = 12.0, 4.2 Hz, 1H), 7.07 (d, J = 4.0 Hz, 2H), 7.00 (d, J = 7.8 Hz, 1H), 3.39 (s, 3H), 1.97 (s, 3H). ¹³C NMR (100 MHz, CDCl₃) δ [ppm] = 161.6, 141.5, 129.2 124.3, 123.9, 117.8, 114.7, 90.1, 29.0, 21.1. IR (neat): 2109, 1680, 1503, 1381 cm⁻¹ HRMS (ESI-TOF) m/z: [M + H − N₂]⁺ calcd for C₁₀H₁₁N₂O₂: 191.0821; found: 191.0820 |
| 5j | 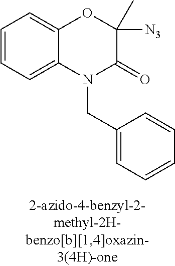 | 46 | White solid; 23.0 mg ¹H NMR (400 MHz, CDCl₃) δ [ppm] = 7.34 (m, 2H), 7.27 (m, 3H), 7.10 (dd, J = 7.7, 1.5 Hz, 1H), 7.04 (m, 1H), 6.99 (m, 1H), 6.89 (m, 1H), 5.48 (d, J = 16.1 Hz, 1H), 4.86 (d, J = 16.1 Hz, 1H), 2.06 (s, 3H). ¹³C NMR (100 MHz, CDCl₃) δ [ppm] = 161.19, 141.7, 135.6, 129.1, 127.7, 126.4, 124.4, 123.9, 118.0, 115.5, 90.2, 45.8, 21.0. IR (neat): 2114, 1697, 1499, 1397, cm⁻¹ HRMS (ESI-TOF) m/z: [M + H − N₂]⁺ calcd for C₁₆H₁₅N₂O₂: 267.1134; found: 267.1125 |

### Example 3

### Continuous Flow Synthesis of Compounds of Formula (I′)

0.1 M solution of 1a′ and a 0.3 M solution of TMSN₃ in DCM were prepared using Scheme 1′ and passed through the 6.6 mm×150 mm Omnifit packed bed reactor (1 g of Amberlyst-15, 6 cm bed height) (Vapourtec R-series) with a flow rate of 0.1 mL/min at a specified temperature. t_{R}=21 min. Compound 3a′ was synthesized using 9-phenyl-9H-fluoren-9-ol (750.0 mg, 2.9 mmol) to afford 9-azido-9-phenyl-9H-fluorene 3a′ (682.0 mg) as a white semi-solid after purification by column chromatography on silica gel directly (EtOAc:hexane=1:99).

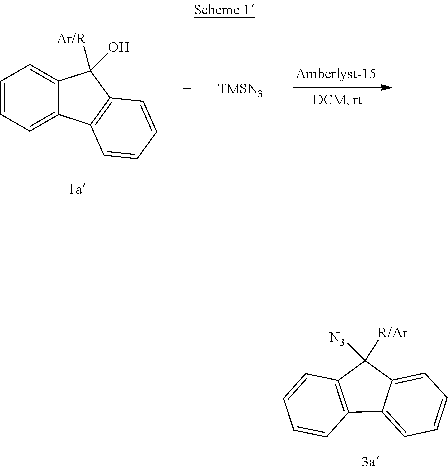

Compounds of the Table 4 were synthesized using the same procedure as recited above.

TABLE 4
Compounds synthesized by continuous flow process

| Name | Structure | % yield* | Characterization data |
|---|---|---|---|
| 3a′ | 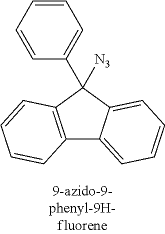 [SMILES: [N-]=[N+]=NC1(c2ccccc2)c2ccccc2-c2ccccc21] | 83 | ¹H NMR (400 MHz, CDCl₃) δ 7.75 (dd, J = 7.6, 0.7 Hz, 2H), 7.44 (m, 2H), 7.31 (m, 9H). ¹³C{¹H} NMR (100 MHz, CDCl₃) δ 147.0, 140.6, 140.2, 129.6, 128.7 (d, J = 2.1 Hz), 127.9, 126.1, 125.2, 120.4, 76.3. IR (neat): 2908 cm⁻¹. HRMS (ESI-TOF) m/z: [M + H − N₂]⁺ calcd for C₁₉H₁₄N: 256.1126; found: 256.1125. |
| 3b′ | 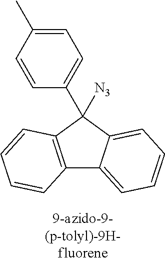 | 89 | ¹H NMR (400 MHz, CDCl₃) δ 7.67 (m, 2H), 7.36 (m, 2H), 7.26 (m, 4H), 7.17 (m, 2H), 7.04 (d, J = 8.0 Hz, 2H), 2.25 (s, 3H). ¹³C{¹H} NMR (100 MHz, CDCl₃) δ 147.1, 140.1, 137.6 (d, J = 2.9 Hz), 129.4 (d, J = 16.2 Hz), 128.6, 126.0, 125.1, 120.4, 76.1, 21.1. IR (neat): 2095 cm⁻¹. HRMS (ESI-TOF) m/z: [M + H − N₂]⁺ calcd for C₂₀H₁₆N: 270.1283; found: 270.1277. |
| 3c′ | 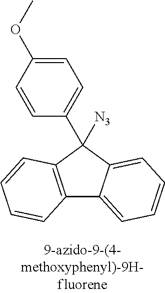 | 97 | ¹H NMR (400 MHz, CDCl₃) δ 7.70 (d, J = 7.3 Hz, 2H), 7.31 (ddd, J = 23.4, 14.5, 6.9 Hz, 8H), 6.80 (d, J = 6.8 Hz, 2H), 3.74 (s, 3H). ¹³C{¹H} NMR (100 MHz, CDCl₃) δ 159.3, 147.2, 140.1, 132.5, 129.5, 129.0, 127.4, 125.1, 120.4, 114.0, 75.9, 55.4. IR (neat): 2092 cm⁻¹. HRMS (ESI-TOF) m/z: [M + H − N₂]⁺ calcd for C₂₀H₁₆NO: 286.1232; found: 286.1230. |
| 3d′ | 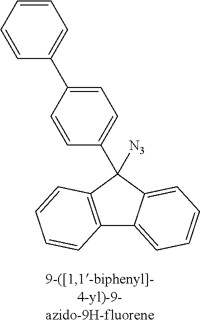 [SMILES: [N-]=[N+]=NC1(c2ccc(-c3ccccc3)cc2)c2ccccc2-c2ccccc21] | 92 | ¹H NMR (400 MHz, CDCl₃) δ 7.77 (d, J = 7.6 Hz, 2H), 7.54 (m, 4H), 7.39 (m, 11H). ¹³C{¹H} NMR (100 MHz, CDCl₃) δ 147.0, 140.8, 140.7, 140.2, 139.6, 129.7, 128.9, 128.7, 127.5, 127.4 127.2, 126.6, 125.2, 120.5, 76.2. IR (neat): 2097 cm⁻¹. HRMS (ESI-TOF) m/z: [M + H − N₂]⁺ calcd for C₂₅H₁₈N: 332.1439; found: 332.1442. |
| 3e′ | 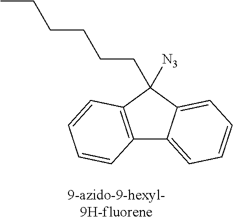 | 92 | ¹H NMR (400 MHz, CDCl₃) δ 7.70 (dd, J = 7.4, 0.6 Hz, 2H), 7.53 (d, J = 7.0 Hz, 2H), 7.40 (m, 4H), 2.15 (m, 2H), 1.17 (m, 6H), 0.94 (m, 2H), 0.82 (t, J = 7.0 Hz, 3H). ¹³C{¹H} NMR (100 MHz, CDCl₃) δ 145.2, 140.4, 129.3, 128.2, 123.8, 120.3, 73.9, 38.3, 31.5, 29.4, 23.9, 22.6, 14.1. IR (neat): 2090 cm⁻¹. HRMS (ESI- TOF) m/z: [M + H − N₂]⁺ calcd for C₁₉H₂₂N: 264.1752; found: 264.1748. |
| 3f′ | 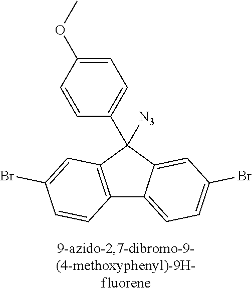 | 92 | ¹H NMR (400 MHz, CDCl₃) δ 7.55 (d, J = 1.1 Hz, 4H), 7.43 (t, J = 1.1 Hz, 2H), 7.19 (m, 2H), 6.84 (m, 2H), 3.79 (s, 3H). ¹³C{¹H} NMR (100 MHz, CDCl₃) δ 159.7, 148.9, 138.0, 133.0, 130.8, 128.5, 127.3, 122.8, 121.9, 114.3, 75.5, 55.4. IR (neat): 2097 cm⁻¹. HRMS (ESI-TOF) m/z: [M + H − N₂]⁺ calcd for C₂₀H₁₄Br₂NO: 441.9442; found: 441.9445. |
| 3g′ | 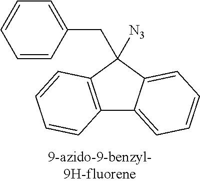 [SMILES: [N-]=[N+]=NC1(Cc2ccccc2)c2ccccc2-c2ccccc21] | 71 | ¹H NMR (400 MHz, CDCl₃) δ 7.61 (d, J = 7.5 Hz, 2H), 7.40 (m, 2H), 7.33 (m, 4H), 7.16 (m, 3H), 6.97 (d, J = 7.6 Hz, 2H), 3.29 (s, 2H). ¹³C{¹H} NMR (100 MHz, CDCl₃) δ 144.6, 140.1, 135.4, 130.9, 129.4, 127.8, 127.7, 126.9, 124.7, 120.3, 73.9, 44.7. IR (neat): 2093 cm⁻¹. HRMS (ESI-TOF) m/z: [M + H − N₂]⁺ calcd for C₂₀H₁₆N: 270.1283; found: 270.1284. |
| 3h′ | 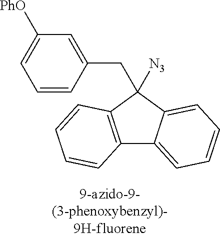 [SMILES: [N-]=[N+]=NC1(Cc2cccc(Oc3ccccc3)c2)c2ccccc2-c2ccccc21] | 90 | ¹H NMR (400 MHz, CDCl₃) δ 7.59 (d, J = 7.5 Hz, 2H), 7.35 (m, 8H), 7.06 (m, 2H), 6.79 (dd, J = 11.4, 8.5 Hz, 3H), 6.63 (d, J = 7.6 Hz, 1H), 6.55 (s, 1H), 3.25 (s, 2H). ¹³C{¹H} NMR (100 MHz, CDCl₃) δ 157.4, 156.3, 144.4, 140.2, 137.3, 129.8, 129.5, 128.9, 127.9, 125.9, 124.6, 123.0, 121.6, 120.3, 118.6, 118.0, 73.9, 44.5. IR (neat): 2094 cm⁻¹. HRMS (ESI- TOF) m/z: [M + H − N₂]⁺ calcd for C₂₆H₂₀NO: 362.1545; found: 362.1540. |
| 3i′ | 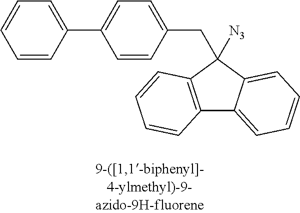 [SMILES: [N-]=[N+]=NC1(Cc2ccc(-c3ccccc3)cc2)c2ccccc2-c2ccccc21] | 98 | ¹H NMR (400 MHz, CDCl₃) δ 7.61 (d, J = 7.5 Hz, 2H), 7.56 (d, J = 1.3 Hz, 2H), 7.36 (m, 11H), 7.02 (d, J = 8.1 Hz, 2H), 3.29 (s, 2H). ¹³C{¹H} NMR (100 MHz, CDCl₃) δ 144.6, 140.8, 140.2, 139.6, 134.6, 131.3, 129.5, 128.9, 127.9, 127.3, 127.1, 126.3, 124.7, 120.4, 73.9, 44.4. IR (neat): 2093 cm⁻¹. HRMS (ESI-TOF) m/z: [M + H − N₂]⁺ calcd for C₂₆H₂₀N: 346.1596; found: 346.1595. |
| 3j′ | 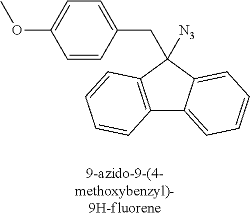 [SMILES: COc1ccc(CC2(N=[N+]=[N-])c3ccccc3-c3ccccc32)cc1] | 83 | ¹H NMR (400 MHz, CDCl₃) δ 7.60 (d, J = 7.5 Hz, 2H), 7.36 (m, 6H), 6.86 (d, J = 8.5 Hz, 2H), 6.66 (d, J = 8.6 Hz, 2H), 3.73 (s, 3H), 3.20 (s, 2H). ¹³C{¹H} NMR (100 MHz, CDCl₃) δ 158.5, 144.7, 140.1, 131.8, 129.4, 127.8, 127.6, 124.7, 120.3, 113.1, 74.0, 55.2, 43.8. IR (neat): 2096 cm⁻¹. |
| 3k′ | 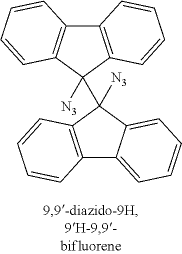 [SMILES: [N-]=[N+]=NC1(C2(N=[N+]=[N-])c3ccccc3-c3ccccc32)c2ccccc2-c2ccccc21] | 35 | ¹H NMR (400 MHz, CDCl₃) δ 7.71 (d, J = 7.5 Hz, 4H), 7.63 (m, 4H), 7.45 (td, J = 7.5, 0.5 Hz, 4H), 7.37 (td, J = 7.5, 1.1 Hz, 4H). ¹³C {¹H} NMR (100 MHz, CDCl₃) δ 141.8, 140.8, 129.6, 128.1, 125.4, 120.4, 64.4. IR (neat): 2091 cm⁻¹. HRMS (ESI-TOF) m/z: [M + H − N₄]⁺ calcd for C₂₆H₁₇N₂: 357.1392; found: 357.1391. |
| 3l′ | 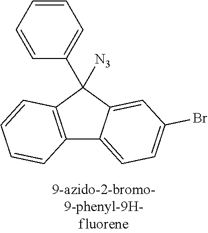 [SMILES: [N-]=[N+]=NC1(c2ccccc2)c2ccccc2-c2ccc(Br)cc21] | 71 | ¹H NMR (400 MHz, CDCl₃) δ 7.72 (d, J = 7.6 Hz, 1H), 7.60 (d, J = 8.1 Hz, 1H), 7.55 (dd, J = 8.1, 1.7 Hz, 1H), 7.44 (m, 2H), 7.35 (dd, J = 7.6, 0.8 Hz, 1H), 7.32 (d, J = 1.6 Hz, 1H), 7.30 (d, J = 5.0 Hz, 5H). ¹³C{¹H} NMR (100 MHz, CDCl₃) δ 149.2, 146.6, 139.8, 139.2, 139.2, 139.1, 132.8, 129.9, 129.1, 128.8, 128.5, 128.2, 126.1, 125.3, 122.3, 121.8, 120.6, 76.0. IR (neat): 2093 cm⁻¹. HRMS (ESI- TOF) m/z: [M + H − N₂]⁺ calcd for C₁₉H₁₃BrN: 334.0231; found: 334.0240. |
| 3m' | 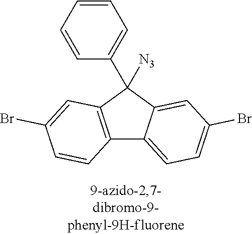 [SMILES: [N-]=[N+]=NC1(c2ccccc2)c2cc(Br)ccc2-c2ccc(Br)cc21] | 78 | ¹H NMR (400 MHz, CDCl₃) δ 7.57 (s, 4H), 7.44 (m, 2H), 7.30 (m, 5H). ¹³C{¹H} NMR (100 MHz, CDCl₃) δ 148.8, 138.9, 138.1, 133.1, 129.0, 128.6, 128.4, 126.0, 122.8, 121.9, 75.8. IR (neat): 2098 cm⁻. HRMS (ESI-TOF) m/z: [M + H − N₂]⁺ calcd for C₁₉H₁₂Br₂N: 411.9336; found: 411.9334. |
| 3n′ | 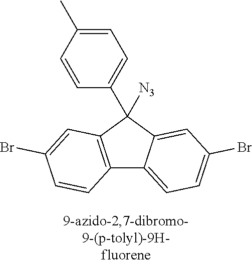 | 79 | ¹H NMR (400 MHz, CDCl₃) δ 7.56 (s, 4H), 7.43 (s, 2H), 7.15 (m, 4H), 2.33 (s, 3H). ¹³C{¹H} NMR (100 MHz, CDCl₃) δ 148.9, 138.3, 138.1, 136.0, 133.0, 129.7, 128.6, 125.9, 122.8, 121.8, 75.7, 21.2. IR (neat): 2096 cm⁻¹. HRMS (ESI- TOF) m/z: [M + H − N₂]⁺ calcd for C₂₀H₁₄Br₂N: 425.9493; found: 425.9487. |

*Mentioned yields are isolated yields

### Example 4: Upscaling of Continuous Flow Reaction with Diphenylmethanol

Diphenylmethanol (1a, 0.1M, 30 mmol, 5.52 gm) in 300 mL dichloromethane+3 equivalents of azidotrimethylsilane (2a, 0.3M, 90 mmol, 10.35 gm) was premixed and flown through the Omnifit® (6.6×150 mm) packed bed column (1 gm of Amberlyst-15, bed height=5 cm, swollen to 6 cm) at room temperature with 0.1 mL/min flow rate with 0-2 bar pressure for 50 hours to afford 29.38 mmol of product (3a). The conversion was monitored by TLC and NMR. After 50 hrs reaction was stopped and reaction mixture was concentrated under vacuum then subjected for column chromatography on silica gel chromatography (hexane). The product 3a was isolated with 6.144 g in 9800 yield with Turnover number (TON)=9.24 and Turnover frequency (TOF)=0.185 h⁻¹. Although the reaction was stopped after 50 h, the catalyst was still active for further reaction. Although, it is known that there is significant loss in activity of Amberlyst-15 for other chemical transformations, negligible loss in activity over a longer period of the reaction was noted for the present disclosure.

### Example 5: Synthesis of Amines Using Staudinger Reaction

To perform the Staudinger reduction step in a flow process (Scheme 3), the stream of 1.0 M solution of (azidomethylene)dibenzene 3a in 3 mL THE was combined in a T-piece with a stream of triphenylphosphine (2M, 2 equiv.) in 3 mL of aqueous THE (THF/water, 9:1) at 0.1 mL/min of 3a and 0.3 mL/min of triphenylphosphine. The resulting mixture was then allowed to react in Vapourtec R-series SS coil reactor (10 mL, 60° C., residence time 25 min) before passing a back-pressure regulator at 5.1 bar and collection in a flask. The volatile component of the crude mixture was evaporated using a vacuum and extracted with DCM. The residue compound 6a was directly purified by silica gel chromatography (EtOAc hexane=40:60). The yield was 58%.

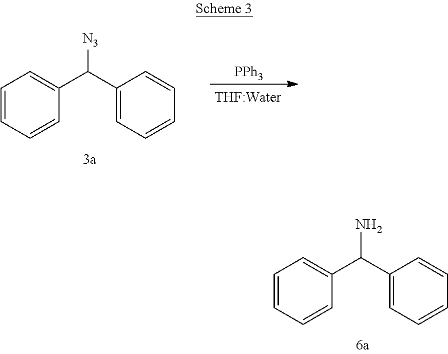 [SMILES: [N-]=[N+]=NC(c1ccccc1)c1ccccc1 ; NC(c1ccccc1)c1ccccc1 ; "PPh3"]

### Example 6: Synthesis of Triazoles Using Click Reaction

[3+2] copper catalyzed alkyne-azide cycloaddition was performed for compound 3a with ethynylbenzene using continuous flow to give the compound 7a (Scheme 4). The azide 3a (0.12 M, 5 mL of t-BuOH:H₂O (1:1)) with 1 mol % CuSO₄·5H₂O, 10 mol % Na ascorbate and ethynylbenzene (0.1 M, 5 mL of t-BuOH:H₂O(1:1)) was flown through pump 1 and pump 2 respectively with 0.1 mL/min flow rate each through PTFE tubing (7 mL) at room temperature with 0.1-0.3 bar pressure. The reaction mixture was collected continuously after 35 min. The reaction mixture was extracted with EtOAc (10 ml×3). The solvent evaporated under vacuum and residue was subjected to column chromatography purification using EtOAc/n-hexane (20:80) to afford the corresponding compound 7a in good yields (73%).

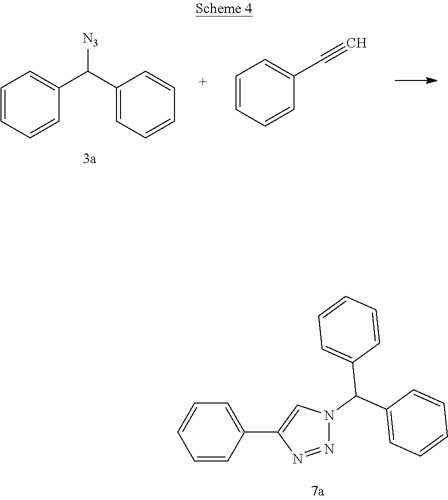

Similar process was used to synthesize a compound 7d from the starting compound 3d in a yield of 62%. Compound 7d is an anti-cancer compound.

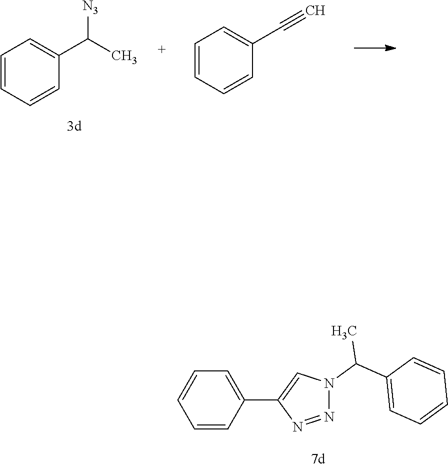

### Example 7: Continuous Flow Synthesis of Quinoxalin-2(1H)-One Derivative

The synthetic utility of compounds of Formula (III) has been demonstrated by synthesis of quinoxalin-2(1H)-one by general scheme 5. The azide of 2-oxindole (Formula (III)) (0.1 M, 5 mL of DMSO) was flown through Vapourtec R Series 10 mL SS coil reactor with a flow rate of 0.1 mL/min at 180° C. at 1.4-2.2 bar pressure. The reaction mixture was collected continuously after 100 min. To the reaction mixture, 50 mL water and 2 mL EtOAc was added and left to precipitate overnight. Next, precipitate formed was filtered, washed with water several times and then dissolved in methanol and passed through a bed of sodium sulphate to afford the corresponding quinoxalin-2(1H)-one derivatives (8) in excellent yields.

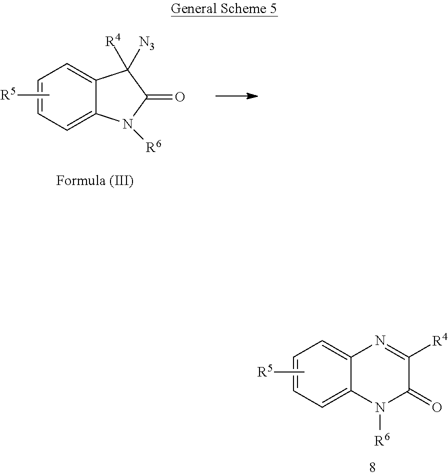

The compounds obtained by the above process are provided in Table 5 synthesized using compounds 3g, 3o, 3p, 3q and 3r of Table 1. Compound 8r can be further converted into an aldose reductase inhibitor in a single step reaction.

TABLE 5
Synthesized quinoxalin-2(1H)-one derivatives

| Name | Structure | % yield* | Characterization data |
|---|---|---|---|
| 8g | 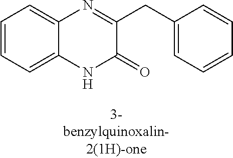 [SMILES: O=c1[nH]c2ccccc2nc1Cc1ccccc1] | 79 | White solid; 55.6 mg ¹H NMR (400 MHz, CDCl₃) δ 11.78 (s, 1H), 7.84 (d, J = 7.7 Hz, 1H), 7.48 (m, 3H), 7.31 (m, 3H), 7.22 (m, 2H), 4.29 (s, 2H). ¹³C NMR (100 MHz, CDCl₃) δ 159.94, 158.3, 156.3, 137.1, 132.9, 131.2, 130.1, 129.7, 129.2, 128.6, 36 126.8, 124.3, 115.6, 40.1. IR (neat): 3800, 2376, 2317, 1743, 1524 cm⁻¹ |
| | | | HRMS (ESI-TOF) m/z: [M + H]⁺ |
| | | | calcd for C₁₅H₁₂N₂O: 237.1028; |
| | | | found: 237.1019. |
| 8o | 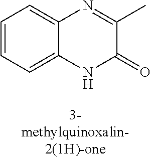 | 83 | White solid; 39.9 mg ¹H NMR (400 MHz, DMSO-d₆) δ 12.30 (s, 1H), 7.67 (m, 1), 7.44 (m, 1H), 7.24 (m, 2H), 2.39 (s, 3H). ³¹C NMR (100 MHz, DMSO-d₆) δ 159.2, 154.9, 131.9, 131.7, 129.3, 127.8, 123.0, 115.2, 20.6. IR (neat): 3392, 2376, 2355, 2318, 2259, 2135, 1651, 1021 cm⁻¹ HRMS (ESI-TOF) m/z: [M + H]⁺ |
| | | | calcd for C₉H₉N₂O: 161.0715; |
| | | | found: 161.0710 |
| 8p | 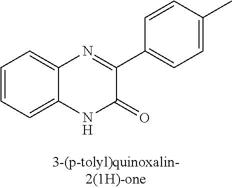 [SMILES: Cc1ccc(-c2nc3ccccc3[nH]c2=O)cc1] | 70 | Yellow solid; 50 mg ¹H NMR (400 MHz, DMSO-d₆) δ 12.46 (m, 1H), 8.26 (m, 2H), 7.82 (dd, J = 8.6, 1.3 Hz, 1H), 7.52 (m, 1H), 7.31 (m, 4H), 2.38 (s, 3H). ¹³C NMR (100 MHz, DMSO-d₆) δ 154.7, 153.8, 140.1, 132.9, 131.9, 130.1, 129.2, 128.6, 123.4, 115.1, 21.1. IR (neat): 3392, 2376, 2352, 2320, 2259, 2135, 1648 cm⁻¹ HRMS (ESITOF) m/z: [M + H]⁺ calcd for C₁₅H₁₃N₂O: 237.1028; |
| | | | found: 237.1019 |
| 8q | 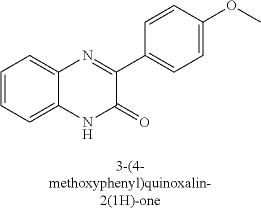 [SMILES: COc1ccc(-c2nc3ccccc3[nH]c2=O)cc1] | 83 | Pale yellow solid; 62.6 mg ¹H NMR (400 MHz, DMSO-d₆) δ 12.52 (s, 1H), 8.39 (m, 2H), 7.80 (m, 1H), 7.50 (ddd, J = 8.3, 7.1, 1.4 Hz, 1H), 7.30 (dd, J = 11.8, 4.4 Hz, 2H), 7.03 (m, 2H), 3.83 (s, 3H). ¹³C NMR (100 MHz, DMSO-d₆) δ 161.0, 154.7, 153.1, 132.1, 131.8, 131.0, 129.8, 128.5, 37 128.2, 123.4, 115.0, 113.4, 55.3 IR (neat): 3741, 2921, 2379, 2315, 1706, 1508 cm⁻¹ HRMS (ESITOF) m/z: [M + H]⁺ |
| | | | calcd for C₁₅H₁₃N₂O₂: 253.0977; |
| | | | found: 253.0975 |
| 8r | 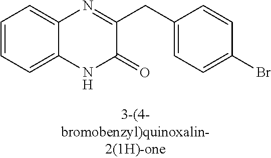 [SMILES: O=c1[nH]c2ccccc2nc1Cc1ccc(Br)cc1] | 81 | White solid; 74.0 mg ¹H NMR (400 MHz, DMSO-d₆) δ 12.42 (s, 1H), 7.70 (d, J = 7.9 Hz, 1H), 7.47 (d, J = 7.1 Hz, 3H), 7.28 (d, J = 8.2 Hz, 4H), 4.09 (s, 2H). ¹³C NMR (100 MHz, DMSO-d₆) δ 159.9, 154.5, 136.9, 132.0, 131.6, 131.2, 129.9, 128.3, 123.2, 119.6, 115.3, 38.4. IR (neat): 2960, 1707, 1422, 1360, |
| | | | 1221, 1092, 979 cm⁻¹. |
| 8i′ | 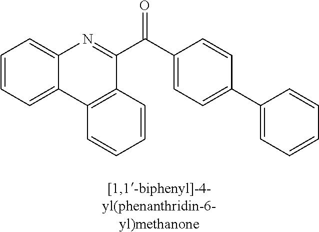 [SMILES: O=C(c1ccc(-c2ccccc2)cc1)c1nc2ccccc2c2ccccc12] | 41 | ¹H NMR (400 MHz, CDCl₃) δ 8.74 (d, J = 8.4 Hz, 1H), 8.67 (m, 1H), 8.25 (dt, J = 5.8, 3.4 Hz, 1H), 8.20 (d, J = 7.8 Hz, 1H), 8.13 (d, J = 8.3 Hz, 2H), 7.91 (dd, J = 8.1, 7.4 Hz, 1H), 7.79 (m, 2H), 7.70 (m, 3H), 7.64 (dt, J = 3.3, 1.9 Hz, 2H), 7.47 (m, 2H), 7.41 (m, 1H). ¹³C{¹H} NMR (100 MHz, CDCl₃) δ 194.5, 157.7, 146.8, 142.8, 140.0, 135.1, 133.5, 131.6, 131.4, 130.8, 129.3, 129.1, 128.5, 128.3, 128.0, 127.52, 127.5, 127.4, 125.4, 124.7, 124.0, 122.5, 122.3. |
| 8j′ | 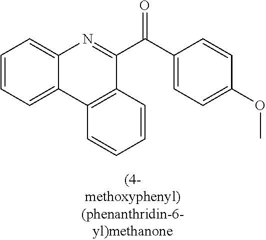 [SMILES: COc1ccc(C(=O)c2nc3ccccc3c3ccccc23)cc1] | 45 | ¹H NMR (400 MHz, CDCl₃) δ 8.72 (d, J = 8.3 Hz, 1H), 8.65 (m, 1H), 8.22 (m, 1H), 8.12 (d, J = 7.8 Hz, 1H), 8.01 (m, 2H), 7.89 (m, 1H), 7.77 (m, 2H), 7.65 (m, 1H), 6.94 (m, 2H), 3.88 (s, 3H). ¹³C{¹H} NMR (100 MHz, CDCl₃) δ 193.6, 164.5, 158.2, 142.9, 133.4, 131.4, 130.7, 129.4, 129.2, 128.2, 127.9, 127.6, 124.6, 124.0, 122.4, 122.3, 114.1, 55.7. IR (neat): 2923, 1613, 1510, 1244 cm⁻¹. HRMS (ESI-TOF) m/z: [M + H]⁺ calcd for C₂₁H₁₆NO₂: 314.1181; found: 314.1178. |
| 8a′ | 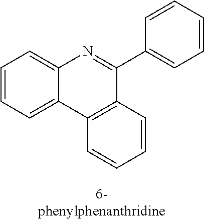 [SMILES: c1ccc(-c2nc3ccccc3c3ccccc23)cc1] | 71 | ¹H NMR (400 MHz, CDCl₃) δ 8.64 (dd, J = 31.5, 8.2 Hz, 2H), 8.27 (d, J = 8.2 Hz, 1H), 8.11 (dd, J = 8.3, 0.5 Hz, 1H), 7.84 (t, J = 7.7 Hz, 1H), 7.76 (m, 3H), 7.68 (m, 1H), 7.57 (m, 4H). ¹³C{¹H} NMR (100 MHz, CDCl₃) δ 161.3, 143.8, 139.8, 133.5, 130.6, 130.4, 129.8, 129.0, 128.9, 128.8, 128.5, 127.2, 127.0, 125.3, 123.8, 122.3, 122.0. IR (neat): 1569, 1358 cm⁻¹. HRMS (ESI-TOF) m/z: [M + H]⁺ calcd for C₁₉H₁₄N: 256.1126; found: 256.1127. |
| 8c′ | 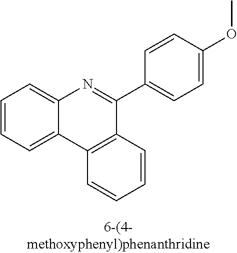 [SMILES: COc1ccc(-c2nc3ccccc3c3ccccc23)cc1] | 77 | ¹H NMR (400 MHz, CDCl₃) δ 8.69 (d, J = 8.3 Hz, 1H), 8.60 (d, J = 7.5 Hz, 1H), 8.20 (m, 2H), 7.84 (m, 1H), 7.68 (m, 5H), 7.10 (m, 2H), 3.92 (s, 3H). ¹³C{¹H} NMR (100 MHz, CDCl₃) δ 161.0, 160.3, 144.0, 133.6, 132.5, 131.3, 130.6, 130.4, 129.1, 128.9, 127.2, 126.8, 125.5, 123.8, 122.3, 122.0, 114.0, 55.6. IR (neat): 1512, 1245 cm⁻¹. HRMS (ESI-TOF) m/z: [M + H]⁺ calcd for C₂₀H₁₆NO: 286.1232; found: 286.1242. |

The foregoing examples are merely illustrative and are not to be taken as limitations upon the scope of the invention. Various changes and modifications to the disclosed embodiments will be apparent to those skilled in the art. Such changes and modifications may be made without departing from the scope of the invention.

### Advantages of the Present Invention

The present disclosure provides a process of synthesizing azides in a safe, mild, reproducible and controlled manner using continuous flow.

The present disclosure provides a process of synthesizing azides that employs non-hazardous, recoverable and recyclable catalyst Amberlyst-15.

### REFERENCES

1. The Chemistry of the Azido Group (Ed.: S. Patai), Wiley, New York, 1971.
2. The Chemistry of Halides, Pseudo-halides and Azides, Supplement D, (Eds.: S. Patai, Z. Rappoport), Wiley, Chichester, 1983.
3. Chemistry of Halides, Pseudo-Halides and Azides, Part 1 (Ed.: S. Patai), Wiley, Chichester, 1995.
4. Chemistry of Halides, Pseudo-Halides and Azides, Part 2 (Ed.: S. Patai), Wiley, Chichester, 1995.
5. Monograph: Azides and Nitrenes Reactivity and Utility (Ed.: E. F. V. Scriven), Academic Press, New York, 1984.
6. Jang, S.; Sachin, K.; Lee, H.; Wook Kim, D.; Soo Lee, H. Development of a Simple Method for Protein Conjugation by Copper-Free Click Reaction and Its Application to Antibody-Free Western Blot Analysis. Bioconjugate Chem. 2012, 23, 2256-2261.
7. Brase, S.; Gil, C.; Knepper, K.; Zimmermann, V. Organic Azides: An Exploding Diversity of a Unique Class of Compounds. Angew. Chem., Int. Ed. 2005, 44, 5188-5240; Padwa, A. Aziridines and Azirines: Monocyclic. In Comprehensive Heterocyclic Chemistry III; Katritzky, A. R., Ramsden, C. A., Scriven, E. F. V., Taylor, R. J. K., Eds.; Elsevier Science: Oxford, 2008; Vol. 1, Chapter 1.01.6.2, pp 50-64.
8. Gololobov, Y. G.; Kasukhin, L. F. Recent Advances in the Staudinger Reaction. Tetrahedron 1992, 48, 1353-1406.
9. (Li, Y. L.; Combs, A. P. Bicyclic Heteroaryl amino alkyl Phenyl Derivatives as PI3K Inhibitors. Int. Patent Appl. WO2015191677A1, Dec. 17, 2015.
10. Kim, M. S.; Yoo, M. H.; Rhee, J. K.; Kim, Y. J.; Park, S. J.; Choi, J. H.; Sung, S. Y.; Lim, H. G.; Cha, D. W. Synthetic Intermediates, Process for Preparing Pyrrolylheptanoic Acid Derivatives There from. Int. Patent Appl. WO2009084827A2, Jul. 9, 2009.
11. Bathula, S. N. V. P.; Vadla, R. Bioactivity of 1, 4-disubstituted 1, 2, 3-triazoles as Cytotoxic Agents against the Various Human Cell Lines. Asian J. Pharm. Clin. Res. 2011, 4, 66-67.
12. Aganda, K. C.; Hong, B.; Lee, A. Visible-Light-Promoted Switchable Synthesis of C-3-Functionalized Quinoxalin-2(1H)-ones. Adv. Synth. Catal. 2021, 363, 1443-1448.
13. Brase, S.; Banert, K., Eds. Organic Azides: Syntheses and Applications; John Wiley & Sons, Ltd.: Chichester, U.K., 2010.
14. Li, J.; Cao, J.; Wei, J.; Shi, X.; Zhang, L.; Feng, J.; Chen, Z. Ionic Liquid Brush as a Highly Efficient and Reusable Catalyst for On-Water Nucleophilic Substitutions. Eur. J. Org. Chem. 2011, 2011, 229-233.
15. Denk, C.; Wilkovitsch, M.; Skrinjar, P.; Svatunek, D.; Mairinger, S.; Kuntner, C.; Filip, T.; Fröhlich, J.; Wanek, T.; Mikula, H. [18F] Fluoroalkyl Azides for Rapid Radiolabeling and (Re)investigation of their Potential Towards in vivo Click Chemistry. Org. Biomol. Chem. 2017, 15, 5976-5982.
16. Kurosawa, W.; Kan, T.; Fukuyama, T. Stereocontrolled Total Synthesis of (−)-Ephedradine A (Orantine). J. Am. Chem. Soc. 2003, 125, 8112-8113.
17. Besset, C.; Chambert, S.; Fenet, B.; Queneau, Y. Direct Azidation of Unprotected Carbohydrates under Mitsunobu Conditions using Hydrazoic Acid. Tetrahedron Lett. 2009, 50, 7043-7047.
18. Terrasson, V.; Marque, S.; Georgy, M.; Campagne, J. M.; Prim, D. Lewis Acid-Catalyzed Direct Amination of Benzhydryl Alcohols. Adv. Synth. Catal. 2006, 348, 2063-2067.
19. Khedar, P.; Pericherla, K.; Kumar, A. Copper Triflate: An Efficient Catalyst for Direct Conversion of Secondary Alcohols into Azides. Synlett 2014, 25, 515-518.
20. Rueping, M.; Vila, C.; Uria, U. Direct Catalytic Azidation of Allylic Alcohols. Org. Lett. 2012, 14, 768-771.
21. Sawama, Y.; Nagata, S.; Yabe, Y.; Morita, K.; Monguchi, Y.; Sajiki, H. Iron-Catalyzed Chemoselective Azidation of Benzylic Silyl Ethers. Chem. Eur. J. 2012, 18, 16608-16611.
22. Reddy, C. R.; Madhavi, P. P.; Reddy, A. S. Molybdenum (V) Chloride-Catalyzed Amidation of Secondary Benzyl Alcohols with Sulfonamides and Carbamates. Tetrahedron Lett. 2007, 48, 7169-7172.
23. Kumar, A.; Sharma, R. K.; Singh, T. V.; Venugopalan, P. Indium (III) Bromide Catalyzed Direct Azidation of α-hydroxyketones using TMSN3. Tetrahedron 2013, 69, 10724-10732.
24. Tummatorn, J.; Thongsornkleeb, C.; Ruchirawata, S.; Thongarama, P.; Kaewmee, B. Convenient and Direct Azidation of Sec-Benzyl Alcohols by Trimethylsilyl Azide with Bismuth (III) Triflate Catalyst. Synthesis 2015, 47, 323-329.
25. Hajipour, A. R.; Rajaei, A.; Ruoho, A. E. A Mild and Efficient Method for Preparation of Azides from Alcohols using Acidic Ionic Liquid [H-NMP] HSO₄. Tetrahedron Lett. 2009, 50, 708-711.
26. Tandiary, M. A.; Masui, Y.; Onaka, M. A Combination of Trimethylsilyl Chloride and Hydrous Natural Montmorillonite Clay: An Efficient Solid Acid Catalyst for the Azidation of Benzylic and Allylic Alcohols with Trimethylsilyl Azide. RSC Adv. 2015, 5, 15736-15739.
27. Kamble, S.; More, S.; Rode, C. Highly Selective Direct Azidation of Alcohols Over a Heterogeneous Povidone-Phosphotungstic Solid Acid Catalyst. New J. Chem. 2016, 40, 10240-10245.
28. Yin, X. P.; Zhu, L.; Zhou, J. Metal-Free Azidation of α-Hydroxy Esters and α-Hydroxy Ketones Using Azidotrimethylsilane. Adv. Synth. Catal. 2018, 360, 1116-1122.
29. Regier, J.; Maillet, R.; Bolshan, Y. A Direct Brønsted Acid Catalyzed Azidation of Benzhydrols and Carbohydrates. Eur. J. Org. Chem. 2019, 2390-2396.
30. Delvillea, M.; Nieuwland, P.; Janssena, P.; Koch, K.; Van Hest, J.; Rutjes, F. Continuous Flow Azide Formation: Optimization and Scale-up. Chem. Eng. J. 2011, 167, 556-559.
31. Sagandira, C.; Watts, P. Safe and Highly Efficient Adaptation of Potentially Explosive Azide Chemistry Involved in the Synthesis of Tamiflu Using Continuous-Flow Technology. Beilstein J. Org. Chem. 2019, 15, 2577-2589.
32. Baxendale, I.; Deeley, J.; Griffiths-Jones, C.; Ley, S.; Saaby, S.; Tranmer, G. A Flow Process for the Multi-Step Synthesis of the Alkaloid Natural Product Oxomaritidine: A New Paradigm for Molecular Assembly. Chem. Commun. 2006, 2566-2568.
33. Donnelly, A.; Zhang, H.; Baumann, M. Development of a Telescoped Flow Process for the Safe and Effective Generation of Propargylic. Molecules 2019, 24, 3658.

We claim:

1. A continuous flow process for synthesizing organic azides of formula (I), a stereoisomer, a tautomer, a pharmaceutically acceptable salt or a pharmaceutically acceptable solvate thereof by direct azidation of alcohols of formula (II), wherein said process comprises the step of:
reacting a compound of formula (II) with trimethylsilyl azide (TMSN3) in Amberlyst-15 catalyst loaded packed-bed reactor at room temperature; 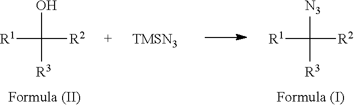
wherein:
R¹ is selected from substituted or unsubstituted (C₆₋₁₆) aryl, or substituted or unsubstituted (C₅₋₁₀) heterocycle;
R² and R³ are independently selected from H, substituted or unsubstituted (C₆₋₁₆)aryl, substituted or unsubstituted (C₁₋₆)alkyl, or substituted or unsubstituted —CH₂—(C₆₋₁₆)aryl; and
the substituent is selected from one or more of halogen, (C₁₋₆) alkyl, cyano, nitro, —NH₂, (C₁₋₆)alkoxy, —COOH, or combinations thereof,
wherein, ratio of the formula (II) to trimethylsilyl azide is 1:3 M,
wherein the Amberlyst-15 and the reactant compounds are present in a ratio of 1:1 w/w,
wherein, flow rate of the reactant compound is 0.08 mL/min to 0.5 mL/min, and
wherein, the process is carried out under a pressure of 0 to 1 bar.

2. The continuous flow process as claimed in claim 1, wherein the compound of formula (I) is selected from the group consisting of: (azidomethylene)dibenzene, 1(azido(phenyl)methyl)-4-chlorobenzene, 4,4′(azidomethylene)bis(methoxybenzene), (1-azidoethyl)benzene, 2-(1-azidoethyl)naphthalene, (1-azidoethane-1,1-diyl)dibenzene, (azidomethanetriyl)tribenzene, 5-(azidomethyl)benzo[d][1,3]dioxole, 5-(azidomethyl)-6-chlorobenzo[d][1,3]dioxole, and 4-(azidomethyl)pyrene or a stereoisomer, a tautomer, a pharmaceutically acceptable salt or a pharmaceutically acceptable solvate thereof.

3. A continuous flow process for synthesizing organic azides of formula (III), a stereoisomer, a tautomer, a pharmaceutically acceptable salt or a pharmaceutically acceptable solvate thereof by direct azidation of alcohols of formula (IV), wherein said process comprises the step of:
reacting a compound of Formula (IV) with trimethylsilyl azide and in Amberlyst-15 catalyst loaded packed-bed reactor at room temperature; 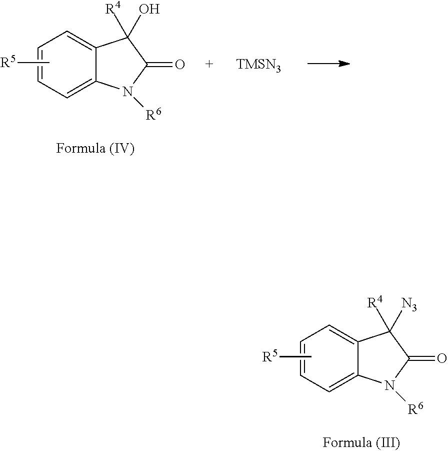
wherein: R⁴, R⁵ and R⁶ are independently selected from one or more of H, halogen, —COOH, nitro, —NH₂, (C₁₋₆)alkoxy, substituted or unsubstituted (C₆₋₁₆)aryl, substituted or unsubstituted (C₁₋₆)alkyl; or substituted or unsubstituted —CH₂—(C₆₋₁₆)aryl; and the substituents is selected from one or more of halogen, (C₁₋₆) alkyl, cyano, nitro, —NH₂, —COOH, (C₁₋₆)alkoxy, or combinations thereof,
wherein, ratio of the formula (IV) to trimethylsilyl azide is 1:3 M,
wherein the Amberlyst-15 and the reactant compounds are present in a ratio of 1:1 w/w,
wherein, flow rate of the reactant compound is 0.08 mL/min to 0.5 mL/min, and
wherein, the process is carried out under a pressure of 0 to 1 bar.

4. The continuous flow process as claimed in claim 3, wherein the compound of formula (III) is selected from the group consisting of: 3-azido-3-methylindolin-2-one, 3-azido-3-phenylindolin-2-one, 3-azido-3-(p-tolyl)indolin-2-one, 3-azido-3-(4-methoxyphenyl)indolin-2-one, 3-azido-3-benzylindolin-2-one, 3-azido-3-(3,4-dimethoxybenzyl)indolin-2-one, 3-azido-3-(4-bromobenzyl)indolin-2-one, 3-azido-3-benzyl-6-chloroindolin-2-one, 3-azido-1,3-dibenzylindolin-2-one, and 3-azido-1,3-dimethylindolin-2-one, or a stereoisomer, a tautomer, a pharmaceutically acceptable salt or a pharmaceutically acceptable solvate thereof.

5. A continuous flow process for synthesizing azides of formula (V), a stereoisomer, a tautomer, a pharmaceutically acceptable salt or a pharmaceutically acceptable solvate thereof by direct azidation of formula (VI), wherein said process comprises the step of:
reacting a compound of formula (VI) with trimethylsilyl azide and in Amberlyst-15 catalyst loaded packed-bed reactor at room temperature; 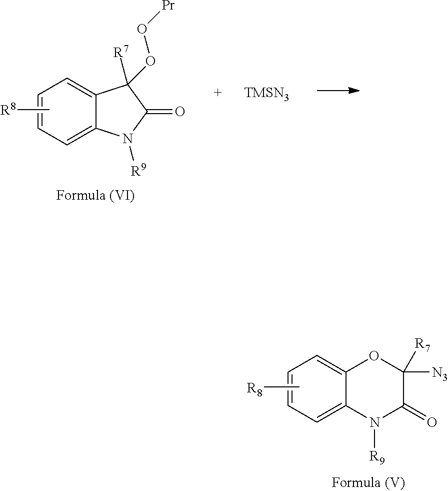
wherein:
R⁷ is selected from H, (C₁₋₆)alkyl, substituted or unsubstituted (C₆₋₁₆)aryl; and substituted or unsubstituted —CH₂—(C₆₋₁₆)aryl;
R⁸ and R⁹ is independently selected from one or more of H, halogen, (C₁₋₆)alkyl, cyano, nitro, (C₁₋₆)alkoxy, substituted or unsubstituted —CH₂—(C₆₋₁₆)aryl, and substituted or unsubstituted (C₆₋₁₆)aryl; and the substituent is selected from one or more of halogen, (C₁₋₆) alkyl, cyano, nitro, (C₁₋₆)alkoxy, —COOH, and —NH₂; and
wherein Pr is a protecting group.
wherein, ratio of the formula (VI) to trimethylsilyl azide is 1:3 M,
wherein the Amberlyst-15 and the reactant compounds are present in a ratio of 1:1 w/w,
wherein, flow rate of the reactant compound is 0.08 mL/min to 0.5 mL/min, and
wherein, the process is carried out under a pressure of 0 to 1 bar.

6. The process as claimed in claim 5, wherein the compounds of Formula (V) is selected from the group consisting of: 2-azido-2-benzyl-2H-benzo[b][1,4]oxazin-3(4H)-one, 2-azido-2-methyl-2H-benzo[b][1,4]oxazin-3(4H)-one, 2-azido-2-(4-methoxyphenyl)-2H-benzo[b][1,4]oxazin-3(4H)-one, 2-azido-2-(2-fluorobenzyl)-2H-benzo[b][1,4]oxazin-3(4H)-one, 2-azido-2-(4-bromobenzyl)-2Hbenzo[b][1,4]oxazin-3(4H)-one, 2-azido-2-benzyl-6-chloro-2Hbenzo[b][1,4]oxazin-3(4H)-one, 2-azido-2-(4-bromobenzyl)-6-chloro-2Hbenzo[b][1,4]oxazin-3(4H)-one, 2-azido-6-chloro-2-(4-methylbenzyl)-2Hbenzo[b][1,4]oxazin-3(4H)-one, 2-azido-2,4-dimethyl-2H-benzo[b][1,4]oxazin-3(4H)-one, 2-azido-4-benzyl-2-methyl-2H-benzo[b][1,4]oxazin-3(4H)-one, a stereoisomer, a tautomer, a pharmaceutically acceptable salt or a pharmaceutically acceptable solvate thereof.

7. A continuous flow process for synthesizing organic azides of formula (I′), a stereoisomer, a tautomer, a pharmaceutically acceptable salt or a pharmaceutically acceptable solvate thereof by direct azidation of alcohols of formula (II′), wherein said process comprises the step of:
reacting a compound of formula (II′) with trimethylsilyl azide and in Amberlyst-15 catalyst loaded packed-bed reactor at room temperature; 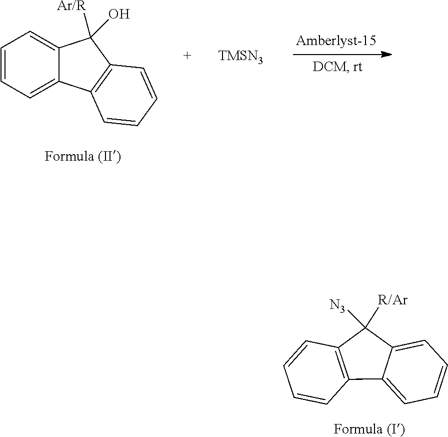
wherein: Ar/R is selected 5 from group consisting of substituted or unsubstituted (C₆₋₁₆)aryl, or substituted or unsubstituted (C₅₋₁₀)heterocycle substituted or unsubstituted (C₆₋₁₆)aryl, substituted or unsubstituted (C₁₋₆)alkyl, or substituted or unsubstituted —CH₂—(C₆₋₁₆)aryl; and
one or more of halogen, (C₁₋₆)alkyl, cyano, nitro, —NH₂, (C₁₋₆)alkoxy, —COOH, or combinations thereof,
wherein, ratio of the formula (II′) to trimethylsilyl azide is 1:3 M,
wherein the Amberlyst-15 and the reactant compounds are present in a ratio of 1:1 w/w,
wherein, flow rate of the reactant compound is 0.08 mL/min to 0.5 mL/min, and
wherein, the process is carried out under a pressure of 0 to 1 bar.

8. The continuous flow process as claimed in claim 7, the compound of formula (I′) is selected from the group consisting of: 9-azido-9-phenyl-9H-fluorene, 9-azido-9-(p-tolyl)-9H-fluorene, 9-azido-9-(4-methoxyphenyl)-9H-fluorene, 9-([1,1′-biphenyl]-4-yl)-9-azido-9H-fluorene,9-azido-9-hexyl-9H-fluorene, 9-azido-2,7-dibromo-9-(4-methoxyphenyl)-9H-fluorene,9-azido-9-benzyl-9H-fluorene, 9-azido-9-(3-phenoxybenzyl)-9H-fluorene, 9-([1,1′-biphenyl]-4-ylmethyl)-9-azido-9H-fluorene, 9-azido-9-(4-methoxybenzyl)-9H-fluorene, 9,9′-diazido-9H,9′H-9,9′-bifluorene, 9-azido-2-bromo-9-phenyl-9H-fluorene, 9-azido-2,7-dibromo-9-phenyl-9H-fluorene and 9-azido-2,7-dibromo-9-(p-tolyl)-9H-fluorene or a stereoisomer, a tautomer, a pharmaceutically acceptable salt or a pharmaceutically acceptable solvate thereof.
